(19) United States
(12) Patent Application Publication
(10) Pub. No.: US 2026/0347832 A1
(43) Pub. Date: Nov. 5, 2026

(54) SYSTEMS AND METHODS FOR UTILIZING A PLASMA MEMBRANE ANCHORED PROTEASE

(71) Applicant: OCTANT, INC., Emeryville, CA (US)

(72) Inventors: Jimin PARK, Emeryville, CA (US); Leon Yen-Lee CHAN, Emeryville, CA (US); Eric Matthew JONES, Emeryville, CA (US); Maris Kealoha KAMALU, Emeryville, CA (US); Henry CHAN, Emeryville, CA (US)

(21) Appl. No.: 19/559,882

(22) Filed: Mar. 6, 2026

Related U.S. Application Data
(63) Continuation of application No. PCT/US2024/045944, filed on Sep. 10, 2024.
(60) Provisional application No. 63/537,762, filed on Sep. 11, 2023.

(51) Int. Cl. C12N 15/10

(52) U.S. Cl. CPC C12N 15/1086; C12N 15/1065

(57) ABSTRACT

Described herein is a system comprising a eukaryotic cell, wherein the eukaryotic cell comprises: a plasma membrane polypeptide coupled to a transcription factor by a linker, wherein the linker comprises a protease cleavable site; and a plasma membrane anchored protease; wherein the plasma membrane anchored protease is capable of cleaving the linker. The system can further include a reporter construct, wherein the transcription factor can bind to a promoter of the reporter construct to indicate an ability of the plasma membrane polypeptide to traffic to the plasma membrane.

### CROSS-REFERENCE

This application is a continuation of International Application No. PCT/US2024/045944, filed internationally on Sep. 10, 2024, which claims the benefit of U.S. Provisional Patent Application No. 63/537,762, filed on Sep. 11, 2023, each of which is incorporated herein by reference in its entirety.

### BACKGROUND

Many human diseases and disorders are associated with suboptimal protein processing, folding, and trafficking. These include, amongst others, Wilson's diseases, Menke's disease, or degenerative diseases such as retinitis pigmentosa, Alzheimer's disease, Parkinson's Disease, Huntington disease, Cystic Fibrosis, Alpha-1 Anti-trypsin Disorder, or Amyotrophic Lateral Sclerosis. There remains a need for compounds that can modulate cell protein processing pathways for the treatment of disease.

### INCORPORATION BY REFERENCE OF SEQUENCE LISTING

The present application is being filed along with a Sequence Listing in electronic format. The Sequence Listing is provided as a file entitled 52652-714601.xml, created Oct. 25, 2024, which is 59,945 bytes in size. The information in the electronic format of the Sequence Listing is incorporated by reference in its entirety.

### SUMMARY

Described herein are systems comprising and uses for plasma membrane proximity reporters for use in screening and identifying test agents that modulate or affect intracellular processing, folding, or trafficking of polypeptides that are destined for the plasma membrane or for secretion. In some embodiments, systems and methods described herein relate to plasma membrane proximity reporters and their activities in response to doses of various test agents. In particular, the systems and methods described herein relate to induction of polypeptide expression or a unique molecular identifier associated with the plasma membrane proximity reporter in response to a dose of one or more test agents. The methods described herein are also suited to identify test agents that modulate intracellular processing, folding, or trafficking of polypeptides in the context of high-throughput assays. The systems and methods described herein can also be used for an amenability assay to evaluate the effect of a test agent on plasma membrane trafficking outcomes for mutant polypeptides, including polypeptides comprising pathogenic mutations identified in patients having certain conditions or diseases (e.g., retinitis pigmentosa, GLP-1R downregulation, cystic fibrosis, etc.).

An aspect of the systems and methods described herein contemplates a system comprising a eukaryotic cell, wherein the eukaryotic cell comprises: a plasma membrane construct comprising a plasma membrane polypeptide coupled to a transcription factor by a linker, wherein the linker comprises a protease cleavage site; and a plasma membrane anchored protease; wherein the plasma membrane anchored protease is capable of cleaving the linker. In some embodiments, the system further comprises a reporter construct comprising a promoter and a reporter gene. In some embodiments, the promoter is bound by the transcription factor upon cleavage of the linker, and wherein the reporter gene is expressed upon cleavage of the linker. In some embodiments, the plasma membrane construct is encoded by an exogenous nucleic acid. In some embodiments, the reporter construct is encoded by an exogenous nucleic acid. In some embodiments, the reporter gene comprises a unique molecular identifier. In some embodiments, the reporter gene comprises a fluorescent protein or a luciferase protein. In some embodiments, the reporter gene comprises a fluorescent protein and a unique molecular identifier or a luciferase protein and a unique molecular identifier. In some embodiments, the plasma membrane anchored protease is integral to the plasma membrane of the eukaryotic cell. In some embodiments, the plasma membrane anchored protease comprises a membrane tethered protease. In some embodiments, the membrane tethered protease comprises a pleckstrin homology domain, platelet-derived growth factor receptor, or Lyn anchor domain. In some embodiments, the transcription factor comprises a DNA binding domain and a transcriptional activation domain. In some embodiments, the DNA binding domain comprises a Gal4, PPR1, Lac9, Zinc Fingers, or LexA DNA binding domain. In some embodiments, the transcriptional activation domain comprises a VP64, VPr, p65, Rta, or VP16 activation domain. In some embodiments, the linker comprises a flexible amino acid linker. In some embodiments, the linker has a length of about 2 to about 31 amino acids. In some embodiments, the plasma membrane anchored protease comprises a tobacco etch virus, aspartic, glutamic, metallo, cysteine, serine, or threonine protease. In some embodiments, the plasma membrane polypeptide comprises rhodopsin. In some embodiments, expression of the plasma membrane construct is inducible. In some embodiments, expression of the plasma membrane construct is induced in response to doxycycline. In some embodiments, the plasma membrane construct localizes at the plasma membrane of the eukaryotic cell after expression of the plasma membrane construct. In some embodiments, the plasma membrane polypeptide comprises an amino acid sequence at least about 90%, 95%, 97%, 98%, 99%, or 100% identical to any one of SEQ IDs 1-6. In some embodiments, expression of the reporter construct indicates an ability of a test agent to alleviate a condition. In some embodiments, the condition is a degenerative disease. In some embodiments, the eukaryotic cell is a mammalian cell. In some embodiments, the mammalian cell is a human cell. In some embodiments, a population of eukaryotic cells comprises the system. Some embodiments contemplate a method of screening a test agent comprising contacting the population of eukaryotic cells to a test agent. In some embodiments, the test agent comprises a small molecule compound.

An aspect of the systems and methods described herein contemplates system comprising a eukaryotic cell, wherein the eukaryotic cell comprises: a plasma membrane construct comprising a plasma membrane polypeptide coupled to a transcription factor by a linker, wherein the linker is cleavable; and a reporter construct comprising a promoter and a reporter gene comprising a unique molecular identifier; wherein the promoter is bound by the transcription factor upon cleavage of the linker, and wherein the reporter gene is expressed upon cleavage of the linker. In some embodiments, the system further comprises a plasma membrane anchored protease capable of cleaving the linker. In some embodiments, the plasma membrane construct is encoded by an exogenous nucleic acid. In some embodiments, the reporter construct is encoded by an exogenous nucleic acid. In some embodiments, the reporter gene further encodes a fluorescent protein or a luciferase protein. In some embodiments, the plasma membrane anchored protease is integral to the plasma membrane of the eukaryotic cell. In some embodiments, the membrane tethered protease comprises a pleckstrin homology domain, platelet-derived growth factor receptor, or Lyn anchor domain. In some embodiments, the transcription factor comprises a DNA binding domain and a transcriptional activation domain. In some embodiments, the DNA binding domain comprises a Gal4, PPR1, Lac9, Zinc Fingers, or LexA DNA binding domain. In some embodiments, the transcriptional activation domain comprises a VP64, VPr, p65, Rta, or VP16 activation domain. In some embodiments, the linker comprises a flexible amino acid linker. In some embodiments, the linker has a length of about 2 to about 31 amino acids. In some embodiments, the plasma membrane anchored protease comprises a tobacco etch virus, aspartic, glutamic, metallo, cysteine, serine, or threonine protease. In some embodiments, the plasma membrane polypeptide comprises rhodopsin. In some embodiments, expression of the plasma membrane construct is inducible. In some embodiments, expression of the plasma membrane construct is induced by doxycycline. In some embodiments, the plasma membrane polypeptide localizes at the plasma membrane of the eukaryotic cell after expression of the plasma membrane polypeptide. In some embodiments, the plasma membrane polypeptide comprises an amino acid sequence at least about 90%, 95%, 97%, 98%, 99%, or 100% identical to any one of SEQ IDs 1-6. In some embodiments, expression of the reporter construct indicates an ability of a test agent to alleviate a condition. In some embodiments, the condition is a degenerative disease. In some embodiments, the eukaryotic cell is a mammalian cell. In some embodiments, the mammalian cell is a human cell. In some embodiments, a population of eukaryotic cells comprises the system. Some embodiments, contemplate a method of screening a test agent comprising contacting the population of eukaryotic cells to a test agent. In some embodiments, the test agent comprises a small molecule compound.

An aspect of the systems and methods described herein contemplates a system comprising a eukaryotic cell, wherein the eukaryotic cell comprises: a plasma membrane construct (PMC) comprising a plasma membrane polypeptide coupled to a transcription factor by a PMC linker, wherein the PMC linker comprises a protease cleavage site; and a plasma membrane anchored protease; wherein the plasma membrane anchored protease is capable of cleaving the PMC linker. In some embodiments, the system further comprises a reporter construct (RC) comprising a RC promoter and a reporter gene. In some embodiments, the RC promoter is bound by the transcription factor upon cleavage of the PMC linker. In some embodiments, the RC promoter comprises a synthetic DNA binding domain responsive promoter. In some embodiments, the RC promoter comprises SEQ ID NO: 43. In some embodiments, the RC promotor comprises a zinc finger binding site. In some embodiments, the RC promoter comprises from 2 to 12 zinc finger binding sites. In some embodiments, the plasma membrane construct is encoded by an exogenous nucleic acid. In some embodiments, the reporter construct is encoded by an exogenous nucleic acid. In some embodiments, the reporter gene comprises a unique molecular identifier. In some embodiments, the reporter gene encodes a fluorescent protein or a luciferase protein. In some embodiments, the reporter gene encodes a fluorescent protein or luciferase protein, and further comprises a unique molecular identifier. In some embodiments, the plasma membrane anchored protease is encoded by an exogenous nucleic acid, optionally wherein expression of the plasma membrane anchored protease is driven by a constitutive promoter. In some embodiments, the plasma membrane anchored protease comprises a membrane tethered protease. In some embodiments, the plasma membrane anchored protease comprises a plasma membrane anchor linked to a protease by a protease tether. In some embodiments, the plasma membrane anchor comprises any one of SEQ ID NOs: 35-38. In some embodiments, the protease tether comprises SEQ ID NO: 39 or 40. In some embodiments, the membrane tethered protease comprises a tobacco etch virus (TEV), aspartic, glutamic, metallo, cysteine, serine, or threonine protease. In some embodiments, the membrane tethered protease comprises a TEV protease or a variant TEV protease, or a functional fragment thereof. In some embodiments, the membrane tethered protease comprises a sequence having at least 90% identity to SEQ ID NO: 41. In some embodiments, the membrane tethered protease comprises SEQ ID NO: 42. In some embodiments, the transcription factor comprises a DNA binding domain and a transcriptional activation domain. In some embodiments, the DNA binding domain comprises a Gal4, PPR1, Lac9, zinc finger, or LexA DNA binding domain. In some embodiments, the DNA binding domain comprises a sequence having at least 90% sequence identity to any one of SEQ ID NOs: 25-29. In some embodiments, the transcriptional activation domain comprises a VP64, VPr, p65, Rta, or VP16 activation domain. In some embodiments, the transcriptional activation domain comprises a sequence having at least 90% sequence identity to any one of SEQ ID NOs: 30-34. In some embodiments, the PMC linker comprises a flexible amino acid linker. In some embodiments, the PMC linker has a length of about 2 to about 31 amino acids. In some embodiments, the PMC linker comprises a TEV-cleavable sequence. In some embodiments, the PMC linker comprises a sequence having at least 90% sequence identity to SEQ ID NO: 20 or 21. In some embodiments, the PMC linker comprises a protease cleavage site comprising at least one of SEQ ID NOs: 22-24. In some embodiments, wherein the plasma membrane polypeptide comprises rhodopsin or a variant thereof. In some embodiments, the plasma membrane polypeptide comprises a sequence having at least 90% sequence identity to any one of SEQ ID NOs: 1-6. In some embodiments, the plasma membrane polypeptide comprises cystic fibrosis transmembrane conductance regulator (CFTR) or a variant of CFTR. In some embodiments, the plasma membrane polypeptide comprises a sequence having at least 90% sequence identity to SEQ ID NO: 7. In some embodiments, the plasma membrane polypeptide comprises a G-protein coupled receptor. In some embodiments, the plasma membrane polypeptide comprises glucagon-like peptide-1 receptor (GLP-1R) or a variant of GLP-1R. In some embodiments, the plasma membrane polypeptide comprises a sequence having at least 90% sequence identity to SEQ ID NO: 8. In some embodiments, the plasma membrane polypeptide comprises a sequence having at least 90% sequence identity to any one of SEQ ID NOs: 9-19. In some embodiments, expression of the plasma membrane construct is inducible. In some embodiments, expression of the plasma membrane construct is induced in response to doxycycline. In some embodiments, the plasma membrane construct localizes at the plasma membrane of the eukaryotic cell after expression of the plasma membrane construct. In some embodiments, expression of the reporter construct indicates an ability of a test agent to alleviate a condition. In some embodiments, the condition is a degenerative disease. In some embodiments, the eukaryotic cell is a mammalian cell. In some embodiments, the mammalian cell is a human cell. In some embodiments, a population of eukaryotic cells comprises a system provided herein. Methods further provided herein include a method of screening a test agent, the method comprising contacting the population of eukaryotic cells comprising a system provided herein to a test agent. In some embodiments, the test agent comprises a small molecule compound.

An aspect of the systems and methods described herein contemplates system comprising a eukaryotic cell, wherein the eukaryotic cell comprises: a plasma membrane construct (PMC) comprising a plasma membrane polypeptide coupled to a transcription factor by a PMC linker, wherein the PMC linker is cleavable; and a reporter construct (RC) comprising a RC promoter and a reporter gene comprising a unique molecular identifier; wherein the RC promoter is bound by the transcription factor upon cleavage of the PMC linker. In some embodiments, the system further comprises a plasma membrane anchored protease capable of cleaving the linker, optionally wherein the plasma membrane anchored protease is encoded by an exogenous nucleic acid. In some embodiments, the plasma membrane construct is encoded by an exogenous nucleic acid. In some embodiments, the plasma membrane construct is encoded by an exogenous nucleic acid. In some embodiments, the reporter construct is encoded by an exogenous nucleic acid. In some embodiments, the reporter gene further encodes a fluorescent protein or a luciferase protein. In some embodiments, the plasma membrane anchored protease is integral to the plasma membrane of the eukaryotic cell. In some embodiments, the plasma membrane anchored protease comprises a plasma membrane anchor linked to a protease by a protease tether. In some embodiments, the plasma membrane anchor comprises any one of SEQ ID NOs: 35-38. In some embodiments, the protease tether comprises SEQ ID NO: 39 or 40. In some embodiments, the membrane tethered protease comprises a tobacco etch virus (TEV), aspartic, glutamic, metallo, cysteine, serine, or threonine protease. In some embodiments, the membrane tethered protease comprises a TEV protease or a variant TEV protease, or a functional fragment thereof. In some embodiments, the membrane tethered protease comprises a sequence having at least 90% identity to SEQ ID NO: 41. In some embodiments, the membrane tethered protease comprises SEQ ID NO: 42. In some embodiments, the transcription factor comprises a DNA binding domain and a transcriptional activation domain. In some embodiments, the DNA binding domain comprises a Gal4, PPR1, Lac9, zinc finger, or LexA DNA binding domain. In some embodiments, the DNA binding domain comprises a sequence having at least 90% sequence identity to any one of SEQ ID NOs: 25-29. In some embodiments, the transcriptional activation domain comprises a VP64, VPr, p65, Rta, or VP16 activation domain. In some embodiments, the transcriptional activation domain comprises a sequence having at least 90% sequence identity to any one of SEQ ID NOs: 30-34. In some embodiments, the PMC linker comprises a flexible amino acid linker. In some embodiments, the PMC linker has a length of about 2 to about 31 amino acids. In some embodiments, the PMC linker comprises a TEV-cleavable sequence. In some embodiments, the PMC linker comprises a sequence having at least 90% sequence identity to SEQ ID NO: 20 or 21. In some embodiments, the PMC linker comprises a protease cleavage site comprising at least one of SEQ ID NOs: 22-24. In some embodiments, the plasma membrane polypeptide comprises rhodopsin or a variant thereof. In some embodiments, the plasma membrane polypeptide comprises a sequence having at least 90% sequence identity to any one of SEQ ID NOs: 1-6. In some embodiments, the plasma membrane polypeptide comprises cystic fibrosis transmembrane conductance regulator (CFTR) or a variant of CFTR. In some embodiments, the plasma membrane polypeptide comprises a sequence having at least 90% sequence identity to SEQ ID NO: 7. In some embodiments, the plasma membrane polypeptide comprises a G-protein coupled receptor. In some embodiments, the plasma membrane polypeptide comprises glucagon-like peptide-1 receptor (GLP-1R) or a variant of GLP-1R. In some embodiments, the plasma membrane polypeptide comprises a sequence having at least 90% sequence identity to SEQ ID NO: 8. In some embodiments, the plasma membrane polypeptide comprises a sequence having at least 90% sequence identity to any one of SEQ ID NOs: 9-19. In some embodiments, expression of the plasma membrane construct is inducible. In some embodiments, expression of the plasma membrane construct is induced by doxycycline. In some embodiments, the plasma membrane polypeptide localizes at the plasma membrane of the eukaryotic cell after expression of the plasma membrane polypeptide. In some embodiments, expression of the reporter construct indicates an ability of a test agent to alleviate a condition. In some embodiments, the condition is a degenerative disease. In some embodiments, the eukaryotic cell is a mammalian cell. In some embodiments, the mammalian cell is a human cell. In some embodiments, a population of eukaryotic cells comprises a system provided herein. Methods further provided herein include a method of screening a test agent, the method comprising contacting the population of eukaryotic cells comprising a system provided herein to a test agent. In some embodiments, the test agent comprises a small molecule compound.

An aspect of the systems and methods described herein contemplates a method of determining amenability for a test agent to rescue defective plasma membrane trafficking of a variant plasma membrane protein of interest, the method comprising: (a) expressing a recombinant form of the variant plasma membrane protein in a host cell and contacting the host cell with the test agent; (b) measuring trafficking of the variant plasma membrane protein to the plasma membrane of the host cell using a system described herein; (c) comparing the trafficking determined in (b) to the trafficking in the host cell when it is not contacted with the test agent, and (d) determining that a patient afflicted with or predisposed to a disease associated with the variant plasma membrane protein is a candidate for treatment with the test agent if the trafficking of the plasma membrane protein is increased in the host cell contacted with the test agent when compared to trafficking in the host cell not contacted with the test agent. In some embodiments, step (d) comprises determining that the patient is a candidate for treatment with the test agent if, in step (c), there is at least a 1.3 to 40 fold increase in trafficking in the host cell contacted with the test agent when compared to trafficking in the host cell not contacted with the test agent. In some embodiments, step (d) comprises determining that the patient is a candidate for treatment with the test agent if the trafficking in the host cell is at least 2% to about 100% of a non-mutant plasma membrane protein. In some embodiments, the test agent is a pharmacological corrector. In some embodiments, the variant plasma membrane protein or a gene encoding the variant plasma membrane protein has been identified from the patient afflicted with or predisposed to a disease associated with the variant plasma membrane protein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 illustrates an exemplary embodiment described herein, wherein a cell comprises a plasma membrane construct including a plasma membrane polypeptide, a linker, and a transcription factor and a plasma membrane proximity reporter encoded by one or more nucleic acid sequences.

FIG. 2 illustrates an exemplary embodiment described herein, wherein the plasma membrane construct is expressed and trafficked to the plasma membrane of the cell.

FIG. 3 illustrates an exemplary embodiment described herein, wherein the plasma membrane construct reaches the plasma membrane of the cell and localizes to the plasma membrane at a polypeptide of the plasma membrane construct.

FIG. 4 illustrates an exemplary embodiment described herein, wherein the plasma membrane construct is cleaved by a plasma membrane anchored protease at a linker liberating a transcription factor, driving expression of a unique molecular identifier by inducing the plasma membrane proximity promoter operatively coupled to the unique molecular identifier.

FIG. 5 illustrates an exemplary process described herein relating to the localization of the plasma membrane construct, cleaving of the plasma membrane construct, expression of a unique molecular identifier read count, and expression of a luciferase gene.

FIG. 6 shows luciferase readout data wherein different cleavage sites are tested.

FIG. 7 shows luciferase readout data wherein the promoter driving plasma membrane anchored protease expression and the plasma membrane anchor are tested.

FIG. 8 shows luciferase readout data wherein the DNA binding domain of the transcription factor is tested.

FIG. 9 depicts Broad Target Scanning to measure effectiveness of different test agents across numerous pathogenic autosomal dominant retinitis pigmentosa variants.

FIG. 10 depicts Deep Mutational Scanning to measure the trafficking consequence of every single possible amino acid change in the rhodopsin protein.

### DETAILED DESCRIPTION

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary embodiments.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems and methods described herein are modular and not limited to sequential steps. Accordingly, terms such as “first” and “second” do not necessarily imply priority, order of importance, or order of acts.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. In some cases, “about” or “approximately refers to an amount that is near (plus or minus) the stated amount by 10%.

Where particular values are described in the application and claims, unless otherwise stated the term “about” should be assumed to mean an acceptable error range for the particular value.

The terms “increased”, “increasing”, or “increase” are used herein to generally mean an increase by a statically significant amount. In some cases, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased”, “decreasing”, or “decrease” are used herein generally to mean a decrease by a statistically significant amount. In some cases, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

As used herein, a “cell” generally refers to a biological cell. A cell is the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), or a cell from a mammal (e.g., a pig, a cow, a goat, a sheep, a rodent, a rat, a mouse, a non-human primate, a human, etc.). Sometimes a cell is not originating from a natural organism (e.g. a cell is a synthetically made, sometimes termed an artificial cell). In some cases, the cell is a primary cell. In some cases, the cell is derived from a cell line.

The term “nucleotide,” as used herein, generally refers to a base-sugar-phosphate combination. A nucleotide comprises a synthetic nucleotide. A nucleotide comprises a synthetic nucleotide analog. Nucleotides is monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP.

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. In some cases, a polynucleotide is exogenous (e.g., a heterologous polynucleotide). In some cases, a polynucleotide is endogenous to a cell. In some cases, a polynucleotide can exist in a cell-free environment. In some cases, a polynucleotide is a gene or fragment thereof. In some cases, a polynucleotide is DNA. In some cases, a polynucleotide is RNA. A polynucleotide can have any three dimensional structure, and can perform any function, known or unknown. In some cases, a polynucleotide comprises one or more analogs (e.g., altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (TRNA), ribosomal RNA (rRNA), short interfering RNA (siRNA), short-hairpin RNA (shRNA), guide RNA (gRNA), micro-RNA (miRNA), non-coding RNA, ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. In some cases, the sequence of nucleotides is interrupted by non-nucleotide components.

The terms “polypeptide” and “protein” are used interchangeably to refer to a polymer of amino acid residues, and are not limited to a minimum length. Polypeptides, including the provided polypeptide chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification. In some cases, the polypeptide encodes a gene or a transgene as described herein.

The term “plasma membrane protein” or “plasma membrane polypeptide” includes, but is not limited to, a transmembrane polypeptide or a polypeptide otherwise targeted to the plasma membrane (e.g., by tethering or anchoring), and includes polypeptides that have proximity to the plasma membrane such that a linker attached to the polypeptide can be cleaved by a plasma membrane anchored protease.

The term “test agent” as described herein refers to any one or more compounds such as a small molecule, peptide, antibody, nucleic acid, siRNA, sequence guided nuclease construct, or gene construct or the like that are introduced to the systems described herein to ascertain an effect of the test agent on the reporter outputs of the system described herein. Not all test agents may introduce a detectable change in the reporter outputs. Test agents also comprise environmental conditions such as pH, temperature, media tonicity and the like.

The term “gene” or “transgene” as used herein refers to a segment of nucleic acid that encodes an individual protein or RNA (also referred to as a “coding sequence” or “coding region”), optionally together with associated regulatory elements such as promoter, operator, terminator and the like, which is located upstream or downstream of the coding sequence. In some cases, the promoter is an inducible promoter. In some embodiments, the regulatory element comprises at least one open reading frame (ORF) that does not encode the transgene. Instead, the ORF in the regulatory element can upregulate the transgene. In some embodiments, the ORF in the regulatory element can downregulate the transgene. In some embodiments, the ORF in the regulatory element is located at a 5′ upstream of the transgene. In some embodiments, the ORF in the regulatory element is located at a 3′ downstream of the transgene. The term “gene” or “transgene” is to be interpreted broadly, and can encompass mRNA, cDNA, CRNA and genomic DNA forms of a gene. In some uses, the term “gene” encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” or “transgene” comprises only the coding sequences (e.g., an “open reading frame” or “coding region”) necessary for encoding a polypeptide. In some aspects, genes or transgenes do not encode a polypeptide, for example, ribosomal RNA genes (rRNA) and transfer RNA (tRNA) genes. In some aspects, the term “gene” or “transgene” includes not only the transcribed sequences, but in addition, also includes non-transcribed regions including upstream and downstream regulatory elements such as regulatory regions, enhancers and promoters. The term “gene” or “transgene” can encompass mRNA, cDNA and genomic forms of a gene.

The term “expression” generally refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. In some embodiment, expression can include biological activity of the polypeptide encoded by the polynucleotide described herein. “Upregulate”, with reference to expression, generally refers to an increased expression level of a polynucleotide (e.g., RNA such as mRNA) and/or polypeptide sequence compared to its expression level in a wild-type state. For example, an expression of gene or transgene can be upregulated by the systems described herein by at least 0.1 fold, 0.2 fold, 0.3 fold, 0.4 fold, 0.5 fold, 1 fold, 2 fold, 3 fold, 4 fold, 5 fold, 10 fold, 20 fold, 50 fold, or more fold compared to an expression of the gene or transgene in a wild-type state (e.g. without the systems described herein upregulating the expression of the gene or transgene). “Downregulate” generally refers to a decreased expression level of a polynucleotide (e.g., RNA such as mRNA) and/or polypeptide sequence compared to its expression in a wild-type state. For example, an expression of gene or transgene can be downregulated by the systems described herein by at least 0.1 fold, 0.2 fold, 0.3 fold, 0.4 fold, 0.5 fold, 1 fold, 2 fold, 3 fold, 4 fold, 5 fold, 10 fold, 20 fold, 50 fold, or more fold compared to an expression of the gene or transgene in a wild-type state (e.g., without the systems described herein upregulating the expression of the gene or transgene). “Wild-type” or “wild-type state” can refer to a phenotype or biological measurements or observations of the expression as it occurs in nature without manipulation by expression vectors or nucleic acids that reduce expression of a target (e.g., expression as a product of a normal allele as opposed to expression as a product of a mutant or engineered gene or by siRNA or a CRISPR/Cas9 system).

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared.

The terms “identity,” “identical,” or “percent identical” when used herein to describe to a nucleic acid sequence, compared to a reference sequence, which can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215:403-410, 1990). Percent identity of sequences can be determined using the most recent version of BLAST, as of the filing date of this application.

The polypeptides of the systems described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In some cases, the vectors comprise regulatory elements such as promoters, enhancers, polyadenylation signals for use in controlling transcription. Regulatory elements can be derived from mammalian, microbial, viral or insect genes. In the embodiments, the vectors comprise the gene expression cassettes described herein. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements for integration.

As used herein the term “transfection” or “transfected” refers to methods that intentionally introduce an exogenous nucleic acid into a cell through a process commonly used in laboratories. Transfection can be effected by, for example, lipofection, calcium phosphate precipitation, viral transduction, or electroporation. Transfection can be either transient or stable.

As used herein the term “transfection efficiency” refers to the extent or degree to which a population of cells has incorporated an exogenous nucleic acid. Transfection efficiency can be measured as a percentage (%) of cells in a given population that have incorporated an exogenous nucleic acid compared to the total population of cells in a system. Transfection efficiency can be measured in both transiently and stably transfected cells.

As used herein a “reporter gene”, “reporter construct”, or equivalents refers to one or more genetic elements in a cell that can be detected using laboratory methods when expressed in the cell. Reporter genes include without limitation luciferase genes, genes encoding fluorescent proteins, gene encoding enzymes (which may act on certain substrates leading to a detectable signal), or unique molecular identifiers or barcode sequence. Reporter genes are generally coupled to a promoter element, response element, or transcription factor binding site making them useful in understanding cell signaling events.

As used herein “reporter activity” refers to the empirical readout from the reporter. For example, a luciferase reporter will have a luminescent readout when incubated with an appropriate substrate. Other reporters like a fluorescent protein may not require a substrate but can be measured via microscopy or a fluorescence plate reader for example. A unique molecular identifier, for example, can be identified by sequencing or an amplification reaction. In some embodiments, the reporter is encoded by a reporter nucleic acid. In some instances, the expression of the reporter is driven by a regulatory element or a promoter described herein. In some embodiment, the expression of the reporter is driven by a cAMP response element such as cAMP response element-binding protein (CREB).

As used herein, “heterologous” or “exogenous” may describe a component of a cell that can be present in or expressed by the cell after introduction, but was originally foreign to the cell. For example, heterologous or exogenous expression of a protein may occur after the introduction of complementary DNA or RNA encoding for a protein of interest into the cell, thus allowing the cell to express the foreign (now heterologous) protein. Heterologous or exogenous also refers to components (e.g., nucleic acids or proteins) that are expressed or are present at higher or lower levels than naturally occurring in the cell, and includes mutant and wild type forms of the components. Heterologous or exogenous also refers to components (e.g., nucleic acids or proteins) that are integrated or expressed in different cellular or genomic locations than naturally present.

As used herein, “operatively coupled” or “operatively linked” components of a cell have one or more activities of the components linked, e.g., when a first activity of a first component occurs, a second activity of a second component occurs. Thus, in effect, when the first activity of the first component, the second activity of the second component follows, making them “operatively coupled” or “operatively linked”. With respect to, for example, transcription factor binding sites or response elements, activation of the response element will lead to expression of a reporter gene downstream of the binding site or response element. As used herein, a “variant” of a polypeptide includes a polypeptide with an amino acid sequence that is different from the amino acid sequence of the polypeptide for the human species, as shown in SEQ ID NOs: 1-6. In general, variants of polypeptides useful for this disclosure are those that have a reduced ability to localize at or traffic to the plasma membrane of the cell. In general, amino acid sequences of variants of polypeptides useful for this disclosure may vary from amino acid sequences of the wild-type of the polypeptide by one or more amino acids. In some embodiments, an amino acid sequence of a variant of a polypeptide varies by one amino acid. In some embodiments, an amino acid sequence an amino acid sequence of a variant of a polypeptide varies by at least one amino acid. In some embodiments, an amino acid sequence an amino acid sequence of a variant of a polypeptide varies by at least one amino acid, at least two amino acids, at least three amino acids, at least four amino acids, at least five amino acids, at least six amino acids, at least seven amino acids, at least eight amino acids, at least nine amino acids, or at least ten amino acids. In some embodiments, an amino acid sequence an amino acid sequence of a variant of a polypeptide varies by one amino acid. In some embodiments, an amino acid sequence an amino acid sequence of a variant of a polypeptide varies by one amino acid, two amino acids, three amino acids, four amino acids, five amino acids, six amino acids, seven amino acids, eight amino acids, nine amino acids, or ten amino acids.

### OVERVIEW

The systems and methods described herein can measure gene regulation activities by evaluating the expression of one or more polypeptides and/or the level of activation of a plasma membrane proximity promoter associated with the polypeptide activities. In this system, a model polypeptide (e.g., rhodopsin) serves as a proxy for intracellular trafficking of polypeptides. This polypeptide may have one or more mutations that result in misprocessing, misfolding, or mis-trafficking. For example, test agents that augment or reduce expression of the polypeptide will influence signaling through a plasma membrane proximity reporter that contains the plasma membrane proximity promoter and which is detectable by a reporter gene induced by a transcription factor. Also described herein are systems and methods for evaluating the effect of a test agent on the expression or trafficking of one or more polypeptides. In some embodiments, the test agent can be a pharmacological corrector (e.g., chaperone). In some embodiments, test agents comprise small molecule compounds. The systems and methods described herein can be used for evaluating the effect of the test agent on treating or alleviating certain conditions or diseases, such as Menke's disease, Wilson's disease, or degenerative diseases such as retinitis pigmentosa, Alzheimer's disease, Parkinson's Disease, Huntington disease, Cystic Fibrosis, Alpha-1 Anti-trypsin Disorder, or Amyotrophic Lateral Sclerosis. The systems and methods described herein can also be used for evaluating the effect of a test agent on increasing plasma membrane expression of a downregulated polypeptide (e.g., glucagon-like peptide-1 receptor (GLP-1R)). The systems and methods described herein can also be used for an amenability assay to evaluate the effect of a particular mutation on plasma membrane trafficking or evaluate the effect of a test agent on plasma membrane trafficking outcomes of mutant polypeptides, including for pathogenic mutations identified in patients having certain conditions or diseases (e.g., retinitis pigmentosa, GLP-1R downregulation, cystic fibrosis, etc.). As described herein, a degenerative disease may include inherited disorders that affect protein trafficking in the plasma membrane. Such degenerative diseases may be chronic diseases.

### Plasma Membrane Activities and Related Polypeptides

Described herein are systems comprising and uses for plasma membrane proximity reporters for use in screening and identifying test agents that modulate or affect intracellular trafficking of polypeptides that are destined for the plasma membrane or for secretion. In some embodiments, systems and methods described herein relate to plasma membrane proximity reporters and their activities in response to doses of one or more test agents. In particular, the systems and methods described herein relate to polypeptide expression or unique molecular identifier expression associated with the plasma membrane proximity reporter in response to one or more test agents. As described herein, “proximity” as used in the “plasma membrane proximity reporter” indicates that expression of the reporter is related to the trafficking of a polypeptide to the plasma membrane of a cell.

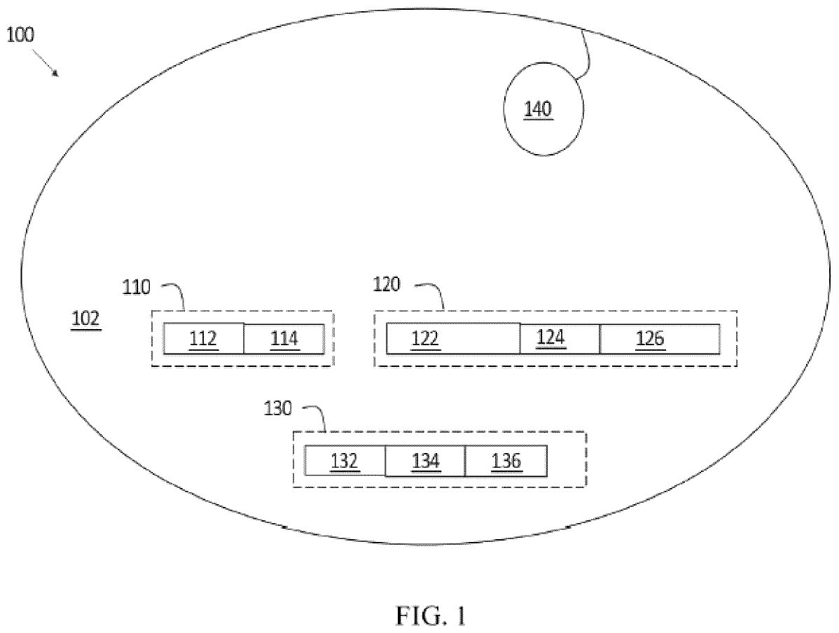

FIG. 1 illustrates example cell 102 of a population of cells 100. In this example, cell 102 includes nucleic acid 110, nucleic acid 120, nucleic acid 130, and protease 140. In some embodiments, the cell 102 may only include one of nucleic acid 110, nucleic acid 120, or nucleic acid 130, or may include a combination thereof. In some embodiments, one or more of nucleic acid 110, nucleic acid 120, and nucleic acid 130 are exogenous to the cell 102. In some embodiments, the cell 102 is a eukaryotic cell. In this depicted embodiment, the protease 140 is anchored to the plasma membrane of the cell.

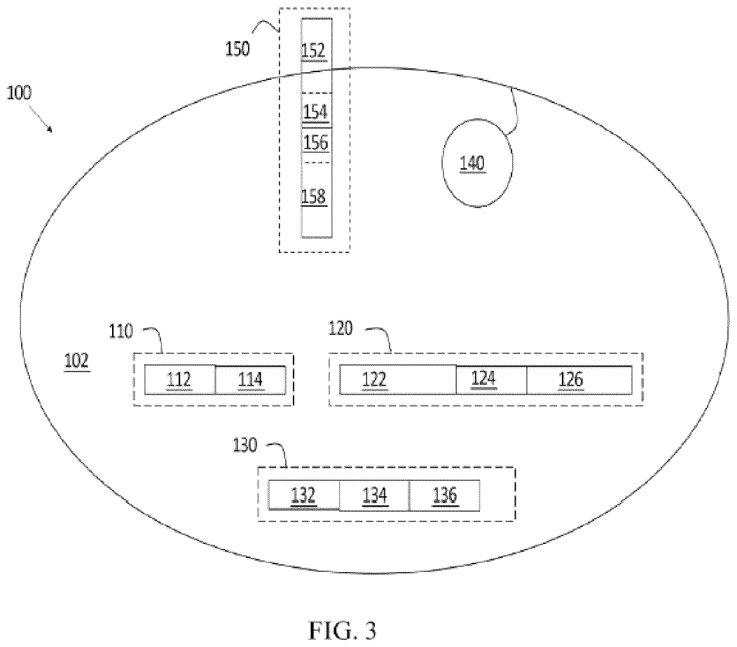

Nucleic acid 110 further includes doxycycline inducible promoter 112 operatively coupled to a coding region for a plasma membrane polypeptide 114. In some embodiments, the doxycycline inducible promoter 112 can instead be a constitutive promoter (e.g., GAPDH or efla). In some embodiments, the plasma membrane polypeptide 114, after being expressed, may be trafficked to the plasma membrane of the cell as part of a plasma membrane construct, which may include one or more components (as described with respect to FIG. 3). In some embodiments, the components may include one or more plasma membrane localizing polypeptides, a transcription factor, and a plasma membrane construct (PMC) linker linking the plasma membrane localized polypeptide and the transcription factor (comprising for example a DNA binding domain and/or a transcriptional activation domain.

In some embodiments, the PMC linker is a flexible amino acid linker. In some embodiments, the PMC linker has a length. In some embodiments, the length of the PMC linker is about 2 amino acids to about 31 amino acids long. In some embodiments, the PMC linker comprises the sequence GSENLYFQSGS (SEQ ID NO: 20) or GSENLYFQYGS (SEQ ID NO: 21). In some embodiments, the PMC linker comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 20 or 21.

In some embodiments, the PMC linker comprises a cleavage site. In some embodiments, the cleavage site is protease-cleavable (e.g., a protease is capable of cleaving the transcription factor from the remainder of a plasma membrane construct including the polypeptide 114 at the cleavage site). In some embodiments, the cleavage site can be cleaved by a TEV protease or variant thereof (e.g., TEV-S219V protease). In some embodiments, the cleavage site comprises the sequence ENLYFQ(x) (SEQ ID NO: 22). In some embodiments, the cleavage site comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 22. In some embodiments, the cleavage site comprises the sequence ENLYFQS (SEQ ID NO: 23) or ENLYFQY (SEQ ID NO: 24). In some embodiments, the cleavage site comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 23 or 24.

In some embodiments, the transcription factor comprises a DNA binding domain (DBD) and a transcriptional activator (also referred to herein as a “activation domain”). In some embodiments, the DNA binding domain of the transcription factor includes a Gal4, PPR1, Lac9, zinc finger, or LexA DNA binding domain. In some embodiments, the DBD comprises at least one of SEQ ID NOs: 25-29. In some embodiments, the DBD comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% sequence identity to any one of SEQ ID NOs: 25-29. In some cases, the DBD binds to a synthetic DBD responsive promoter (e.g., YB_tata). In some embodiments, the DBD binds to a sequence having about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 43.

In some embodiments, the transcriptional activator (i.e., transactivator) comprises a VP64, VPr, p65, Rta, or VP16 activation domain. In some embodiments, the transactivator comprises at least one of SEQ ID NOs: 30-34. In some embodiments, the transactivator comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% sequence identity to any one of SEQ ID NOs: 30-34.

In some embodiments, the polypeptide 114 is rhodopsin or a variant of rhodopsin (e.g., Rhodopsin-P23H). In some embodiments, the polypeptide 114 comprises a sequence having about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NOs: 1-6. In some embodiments, the polypeptide 114 is cystic fibrosis transmembrane conductance regulator (CFTR) or a variant thereof. In some embodiments, the polypeptide 114 comprises a sequence having about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 7. In some embodiments, the polypeptide 114 is a G-protein coupled receptor (e.g., GLP-1R) or a variant of a G-protein coupled receptor. In some embodiments, the polypeptide 114 comprises a sequence having about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NOs: 8-19. The plasma membrane construct including polypeptide 114 can be a variant of a wildtype plasma membrane polypeptide. In some embodiments, the plasma membrane construct including polypeptide 114 comprises a variant or mutant of any one of SEQ ID NOs: 1-19.

Nucleic acid 120 further includes a plasma membrane proximity reporter comprising a plasma membrane proximity promoter 122 that can be bound and activated by the transcription factor linked to the plasma membrane polypeptide 114 operatively coupled to a reporter gene. In this depicted embodiment, the reporter gene includes luciferase gene 124 and unique molecular identifier 126. As described herein, a unique molecular identifier may be referred to as a “UMI” or “barcode”. In some embodiments, the reporter gene, and by extension the nucleic acid 120, does not include luciferase gene 124 and the plasma membrane proximity promoter is only operatively coupled to the unique molecular identifier 126. In some embodiments, the reporter gene encodes a fluorescent protein instead of the luciferase gene 124. In some embodiments, the reporter gene encodes both a fluorescent protein and the luciferase gene 124. In some embodiments, the plasma membrane proximity promoter 122 may be an inducible promoter.

In some embodiments, the plasma membrane proximity promoter comprises a synthetic DBD responsive promoter (e.g., YB_tata). In some embodiments, the plasma membrane proximity promoter comprises SEQ ID NOs: 43. In some embodiments, the plasma membrane proximity promoter comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 43. In some embodiments, the plasma membrane proximity promoter comprises at least 1, 2, 3, 4, 5, 6, 7, 8, 9, 10, 11, or 12 zinc finger binding sites. In some embodiments, the plasma membrane proximity promoter comprises from about 1 to 12 zinc finger binding sites. In some embodiments, the plasma membrane proximity promoter is configured to bind a sequence comprising SEQ ID NO: 25. In some embodiments, the plasma membrane proximity promoter is configured to bind a sequence having at least about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 25.

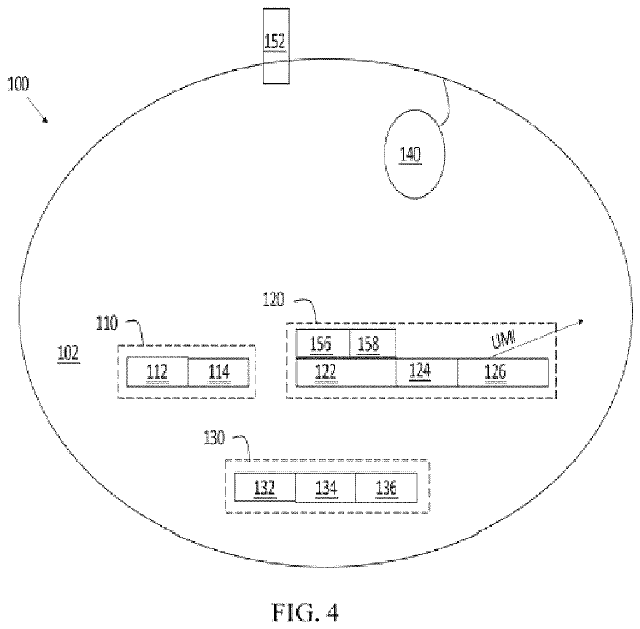

Nucleic acid 130 encodes a plasma membrane anchored protease. Nucleic acid 130 further includes promoter 132 operatively coupled to a gene encoding a plasma membrane anchor 134 and a protease 136. In some embodiments, the promoter 132 may be a constitutive promoter (e.g., GAPDH or efla) or an inducible promoter. In some embodiments, when the promoter 132 is activated, the plasma membrane anchor 134 and the protease 136 may be expressed. In some embodiments, the expressed protease is the same type of protease as the protease 140. In some embodiments, the expressed plasma membrane anchor 134 may anchor the expressed protease to the plasma membrane. In some embodiments, the expressed plasma membrane anchor 134 comprises a transmembrane or plasma membrane localizing domain. In some embodiments, the plasma membrane anchor 134 encodes a platelet-derived growth factor receptor, Lyn (e.g., Lyn11), or Pleckstrin Homology (PH) domain polypeptide or a variant thereof (e.g., PHDelta1, PHDelta3). In some embodiments, the plasma membrane anchor comprises a sequence having about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 35-38. In some embodiments, the nucleic acid 130 further comprises a sequence encoding for a protease tether between the plasma membrane anchor 134 and the protease 136. The protease tether can comprise a flexible amino acid linker. In some embodiments, the protease tether has a length of about 2 to about 31 amino acids. In some embodiments, the protease tether comprises the sequence ASPSNPGASNGS (SEQ ID NO: 39) or GGGGSGGGGS (SEQ ID NO: 40). In some embodiments, the protease tether comprises a sequence having at least 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 39 or 40. In some embodiments, the protease 136 comprises a sequence having about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 41 or 42. When targeted or localized to the plasma membrane, the protease may be capable of cleaving a linker joining the plasma membrane localizing polypeptide of a transcription factor at a cleavage site (as described further with respect to FIGS. 3-4).

In some embodiments, the cell may be present in an environment with increased doxycycline concentrations. In some embodiments, doxycycline may be introduced to the cell, thus increasing activation rates of the doxycycline inducible promoter 112 and increasing expression of the plasma membrane polypeptide 114 bound to the doxycycline inducible promoter. In some embodiments, the doxycycline inducible promoter 112 comprises a sequence having about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 44. In some embodiments, increased expression of the plasma membrane polypeptide 114 within the population of cells 100 following introduction of doxycycline results in the plasma membrane polypeptide 114 reaching the plasma membrane of the cell. In some embodiments, the plasma membrane polypeptide may not reach the plasma membrane or may reach the plasma membrane at a reduced rate compared to a wild-type version in a wild-type cell (e.g., the plasma membrane polypeptide comprises one or more variants that disrupts folding, processing, or trafficking). In some embodiments, the presence of a test agent within the cell 102 or contact of a test agent to the cell 102 may result in increased ability of the expressed plasma membrane polypeptide 114 to localize to the plasma membrane of the cell 102.

In this depicted example, cell 102 further includes protease 140. In this depicted embodiment, the protease 140 is anchored to the plasma membrane of the cell. In some embodiments, the protease is capable of cleaving a linker of the plasma membrane construct that includes the plasma membrane polypeptide 114. In some embodiments, the protease 140 is an integral to the plasma membrane of the cell. For example, the protease may enter the cell through the secretory pathways of the cell or may reside in the secretory pathways of the cell. In some embodiments, the protease 140 comprises a transmembrane domain. In some embodiments, the protease 140 is anchored or localized near the plasma membrane by a plasma membrane anchor. In some embodiments, the plasma membrane anchor comprises a domain from a platelet-derived growth factor receptor, Lyn (e.g., Lyn11), Pleckstrin Homology domain (e.g., PHDelta1 or PHDelta3 polypeptide), or a variant thereof. In some embodiments, the protease 140 is anchored or localized near the plasma membrane by a platelet-derived growth factor receptor, Lyn (e.g., Lyn11), PHDelta1, or PHDelta3 polypeptide, or a functional fragment thereof, or a variant thereof. In some embodiments, the plasma membrane anchor comprises a sequence having about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 35-38. In some embodiments, the protease 140 comprises a tobacco etch virus, aspartic, glutamic, metallo, cysteine, serine, or threonine protease. In some embodiments, the protease 140 comprises a sequence having about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 41 or 42. An optional protease tether between the plasma membrane anchor and the protease can be included. In some embodiments, the protease tether comprises the sequence ASPSNPGASNGS (SEQ ID NO: 39) or GGGGSGGGGS (SEQ ID NO: 40). In some embodiments, the protease tether comprises a sequence having about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 39 or 40.

As noted above, while in this depicted embodiment the nucleic acid 110 comprises the doxycycline inducible promoter 112 and the coding region for the plasma membrane polypeptide 114, the nucleic acid 120 comprises plasma membrane proximity promoter 122, operably coupled to a luciferase gene 124, and/or UMI 126, and the nucleic acid 130 comprises a promoter 132 operatively coupled to a gene encoding the plasma membrane anchor 134, and protease 136, other embodiments of nucleic acids 110, 120, and 130 may have different constructs or may not be present. For example, in some embodiments, the nucleic acid 120 may include luciferase gene 124 but not UMI 126. In other embodiments, the nucleic acid 120 may include UMI 126 but may not include luciferase gene 124. In some embodiments, nucleic acid 110 may not be present. In some embodiments, nucleic acid 130 may not be present. In some embodiments, neither nucleic acid 110 nor nucleic acid 130 may be present. In some embodiments, a different reporter gene such as beta-galactosidase, beta-lactamase, alkaline phosphatase, RFP (red fluorescence protein), and/or GFP (green fluorescence protein) may be operatively coupled to the plasma membrane proximity promoter 122 instead of a luciferase gene 124. While certain constructs are described above, these constructs are exemplary and other constructs may be used. Even further, in other embodiments, various features of nucleic acid 110, nucleic acid 120, and nucleic acid 130 from this depicted example (e.g., doxycycline inducible promoter 112 and/or coding region for plasma membrane polypeptide 114 of nucleic acid 110, plasma membrane proximity promoter 122, luciferase gene 124, and/or UMI 126 of nucleic acid 120, and promoter 132, plasma membrane anchor 134, and protease 136 of nucleic acid 130) may be included on a plurality of nucleic acids instead of a single nucleic acid (e.g., each of nucleic acid 110, nucleic acid 120, and/or nucleic acid 130). Alternatively, in other embodiments, various features of nucleic acid 110, nucleic acid 120, and nucleic acid 130 may be included on a single nucleic acid (e.g., doxycycline inducible promoter 112 and/or coding region for plasma membrane construct 114 of nucleic acid 110, plasma membrane proximity promoter 122, luciferase gene 124, and/or UMI 126 of nucleic acid 120, and promoter 132, plasma membrane anchor 134, and protease 136 of nucleic acid 130 all included on a single nucleic acid).

In some embodiments, the doxycycline promoter 112 may be induced by a dose of doxycycline. In some embodiments, the plasma membrane proximity promoter 122 may be induced by a transcription factor cleaved from the plasma membrane polypeptide 114.

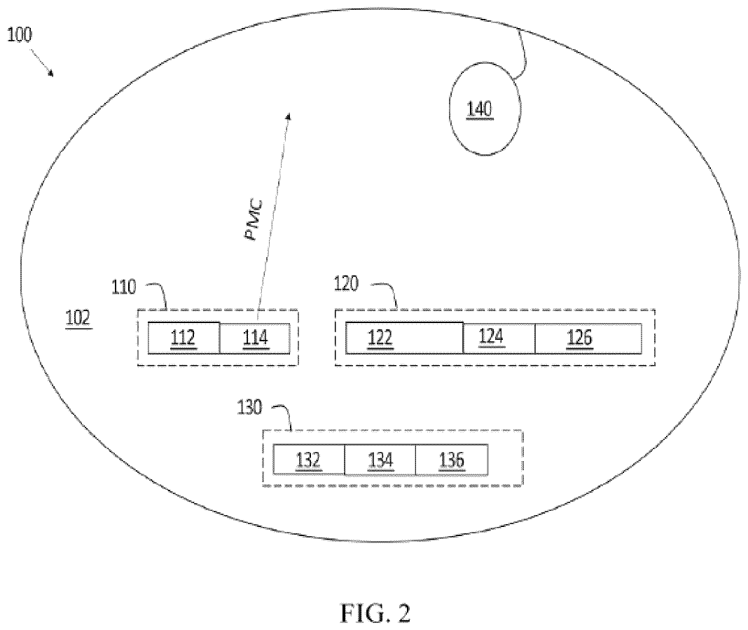

FIG. 2 illustrates example cell 102 expressing the plasma membrane polypeptide 114. In this depicted embodiment, the cell 102 has been contacted with doxycycline. In other embodiments, doxycycline may not be introduced to the cell 102. The presence of the doxycycline inducible promoter 112 in nucleic acid 102 of cells in the population of cells 100 leads to greater expression of the plasma membrane polypeptide 114 when the population of cells is in the presence of doxycycline, which in turn can lead to a greater presence of the plasma membrane polypeptide 114 at the plasma membrane of the cell 102. In embodiments without the doxycycline inducible promoter 112, the plasma membrane polypeptide 114 may still be expressed, but expression of the plasma membrane polypeptide 114 may be reduced as compared to cells that contain the doxycycline inducible promoter 112. Thus, in this depicted embodiment where the cell 102 has been contacted with doxycycline, the doxycycline inducible promoter 112 is induced and expresses plasma membrane polypeptide 114.

In some embodiments, the expression of the plasma membrane polypeptide 114 is associated with one or more diseases, such as retinitis pigmentosa, Alzheimer's disease, Parkinson's Disease, Huntington disease, Cystic Fibrosis, Alpha-1 Anti-trypsin Disorder, or Amyotrophic Lateral Sclerosis. In particular, the one or more diseases may occur when there is a deficiency of one or more polypeptides included in the plasma membrane polypeptide 114 reaching the plasma membrane of a person's cells (e.g., cell 102). In some cases, even when the polypeptides are present in the person's cells, the polypeptides are not transferred to the plasma membrane of the cell due to a deficiency of the polypeptide or variant of the polypeptide, or a deficiency in a related trafficking pathway of the cell. In some cases, even when the plasma membrane polypeptide 114 is expressed, the plasma membrane polypeptide 114 which includes the polypeptide may not reach the plasma membrane of the cell (e.g. due to a mutation of the plasma membrane polypeptide or a deficiency in cellular processing pathways for the plasma membrane polypeptide.

Thus, in some embodiments, a test agent may be present in the cell to aid in trafficking of the plasma membrane polypeptide 114 to the plasma membrane of cell 102. In some embodiments, the test agent may be exogenously added to the cell 102. In some embodiments, the test agent may be contacted to the population of cells 100. In some embodiments, the presence of the test agent in the cell 102 may increase the rate at which the plasma membrane construct 114 is trafficked to the plasma membrane of the cell. In some embodiments, the presence of the test agent in the cell 102 may increase the rate at which the one or more polypeptides is present at the plasma membrane of the cell after being expressed.

Thus, by both expressing the plasma membrane polypeptide 114 and increasing the rate of the plasma membrane polypeptide 114 reaching the plasma membrane of the cell, the above described systems and methods may be useful in identifying test agents useful for treating a disease associated with misfolded or mis-trafficked polypeptides.

FIG. 3 illustrates example cell 102 where plasma membrane construct 150 containing an expressed plasma membrane polypeptide 152 (e.g., expressed by nucleic acid 110) has reached the plasma membrane of cell 102. In this depicted example, as described above, the expressed plasma membrane construct 150 includes a plasma membrane polypeptide 152, linker 154, and a transcriptional factor comprising DNA binding domain 156, and transcriptional activator 158. In some embodiments, the polypeptide 152 is rhodopsin or a variant of rhodopsin. In some embodiments, a variant of rhodopsin may be the RhoP23H variant, hRHO variant, p.G90D variant, p. T941 variant, p.E113K variant, p.A292E variant, p.A295V variant, or other variants of rhodopsin.

In this depicted embodiment, the linker 154 includes a cleavage site which is protease-cleavable. Additionally, the protease 140 is capable of cleaving the linker at the cleavage site. Upon cleaving the linker at the cleavage site, the transcriptional factor is released into the cell, while the polypeptide 152 remains at the plasma membrane of the cell. The polypeptide 152 of the plasma membrane construct 150 may remain at the plasma membrane of the cell 102 after the linker 154 is cleaved.

FIG. 4 illustrates example cell 102 where the transcription factor including the DNA binding domain 156 and the transcriptional activator 158 bind to nucleic acid 120 while the polypeptide 152 remains at the plasma membrane of the cell 102 after the linker 154 has been cleaved. As described above with respect to FIG. 3, the linker 154 is cleaved, allowing the polypeptide 152 to remain localized at the plasma membrane of the cell while the DNA binding domain 156 and the transcriptional activator 158 are released into cell 102. In this depicted example, the DNA binding domain 156 and transcriptional activator 158 bind to and activate transcription at nucleic acid 120 at the plasma membrane proximity promoter 122. In some embodiments, the DNA binding domain 156 and the transcriptional activator 158 form a chimeric transcription factor (e.g., derived from different naturally occurring transcription factors). In some embodiments, the DNA binding domain 156 and the transcriptional activator 158 are a synthetic transcription factor (e.g., either DNA binding domain and/or transcriptional activator comprise non naturally occurring sequences).

When the DNA binding domain 156 binds to the nucleic acid 120 at plasma membrane proximity promoter 122, expression of one or more components of the nucleic acid 120 is induced. In some embodiments, expression of the UMI 126 is induced. In some embodiments, expression of luciferase gene 124 is induced. In some embodiments, expression of both the UMI 126 and luciferase gene 124 is induced. After expression of the UMI 126 and/or luciferase gene 124, a read count of the UMI 126 and/or luciferase gene 124 may be determined for the plurality of cells 100 (i.e., through sequencing of the UMI in a downstream process).

Thus, as described through FIGS. 1-4, the processes of cell 102 and similar cells in plurality of cells 100 help to determine how those diseases or conditions are being alleviated by use of a test agent. For example, the gene encoding the plasma membrane polypeptide 114 of FIGS. 1-4 and the plasma membrane construct 150 of FIG. 3 include a polypeptide (e.g., polypeptide 152) that is prone to misfolding or mis-trafficking and may be associated with the certain diseases or conditions (e.g., rhodopsin, which is associated with degenerative diseases such as retinitis pigmentosa). In many cases, the certain diseases or conditions are caused by an inability of the polypeptide to reach the plasma membrane of the cell. Other polypeptides may be encoded by the gene encoding the plasma membrane polypeptide 114 including, for example, the Cystic fibrosis transmembrane conductance regulator or the alpha-1 antitrypsin enzyme.

However, upon inclusion of a test agent that increases trafficking of the plasma membrane polypeptide to the plasma membrane, the expressed polypeptide has an increased ability to reach the plasma membrane of the cell. The ability of the test agent in increasing the trafficking of the plasma membrane polypeptide can then be measured by read counts of an expressed UMI or activity of an expressed luciferase gene that is expressed by the plurality of cells 100. For example, after the plasma membrane polypeptide reaches the plasma membrane of the cell, the plasma membrane construct comprising the plasma membrane polypeptide may then be cleaved at a linker (e.g., the plasma membrane construct linker 154 of FIG. 3) by proteases anchored to the plasma membrane of the cells that may be capable of cleaving the linker. In some embodiments, the cells may express the protease and/or plasma membrane anchor to be localized at the plasma membrane endogenously. In other embodiments, the protease may be supplied on an exogenous nucleic acid as depicted Upon cleavage, the polypeptide may remain localized to the plasma membrane, while a transcription factor including a DNA binding domain and a transcriptional activator of the plasma membrane construct is released into the cell.

The transcription factor may then bind to a plasma membrane proximity promoter, which in turn induces expression of a UMI and/or a luciferase gene by the activation domain. For example, a read count of the UMI in the plurality of cells indicates the amount of expression of the UMI, which is related to the activation of the reporter gene by the transcription factor in the plurality of cells. Further, the activation of the reporter gene (e.g., UMI, luciferase, fluorescent protein, etc.) in the plurality of cells is related to the rate at which the protease anchored to the plasma membrane cleaves the plasma membrane construct including the plasma membrane polypeptide after it localizes at the plasma membrane of the cell. The rate at which the protease anchored to the plasma membrane of the cell cleaves the plasma membrane construct is further indicative of the rate at which the plasma membrane polypeptide localizes at the plasma membrane of the cell where the polypeptide that alleviates the certain diseases or conditions can bind.

Thus, by measuring the UMI read count in a downstream sequencing assay, the rate at which the plasma membrane polypeptide localizes at the plasma membrane can be measured, which indicates an ability to alleviate or prevent the certain diseases or conditions by a test agent or test condition applied to the cells. Further, the plurality of cells can be contacted with a test agent in order to determine the ability of the test agent to increase or decrease the trafficking of the plasma membrane construct including the polypeptide, and therefore, determine if the test agent may aid in alleviating or preventing the certain diseases or conditions. By running multiple assays with different test agents contacted to the plurality of cells, an ideal test agent for alleviating or preventing the certain diseases or conditions may then be determined.

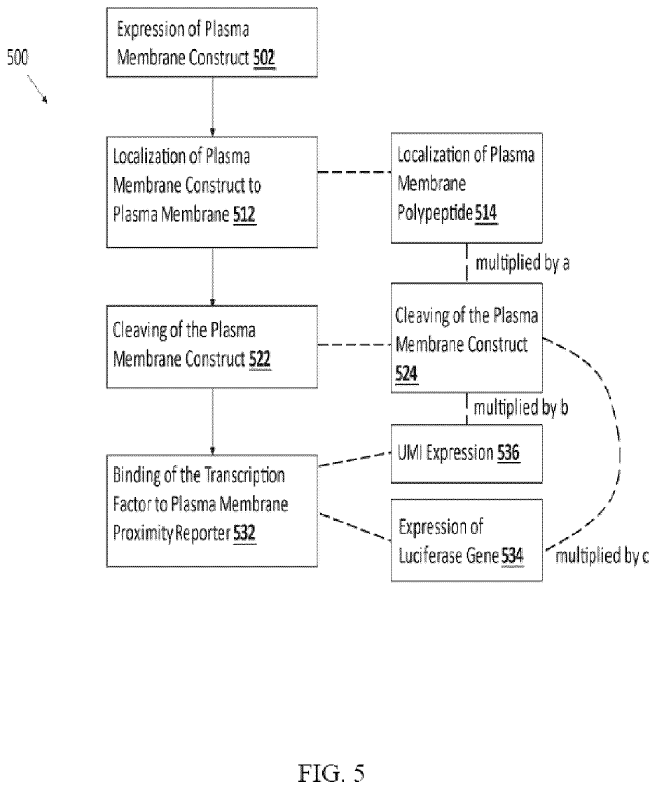

FIG. 5 illustrates example process 500 showing steps 502, 512, 522, and 532, which indicate the relationship between the rate of localization of plasma membrane construct 514 (e.g., the rate at which an expressed plasma membrane polypeptide 114 of FIGS. 1-2 localizes to the plasma membrane of a cell), the rate of cleaving of the plasma membrane construct (e.g., plasma membrane polypeptide 150 of FIG. 3 which may contain polypeptide 152 of FIG. 3) by an anchored protease (e.g., anchored plasma membrane protease 140 of FIGS. 1-4), the expression 536 of a UMI (e.g., UMI 126 of FIGS. 1-4), and/or the expression 534 of a luciferase gene (e.g., luciferase gene 124 of FIGS. 1-4).

For example, the process starts with step 502, where a promoter expresses a plasma membrane polypeptide. In some embodiments, plasma membrane polypeptides localize to the plasma membrane after being expressed (e.g., polypeptide 152 of FIGS. 3-4 localizing to the plasma membrane). The expressed plasma membrane polypeptide may be part of a plasma membrane construct, which further includes a linker with a cleavage site (e.g., the plasma membrane construct linker 154 of FIG. 3), and a transcription factor include a DNA binding domain (e.g., DNA binding domain 156 of FIGS. 3-4) and/or a transcriptional activator (e.g., transcriptional activator 158 of FIGS. 3-4). In some embodiments, the promoter may be inducible or may be constitutive. If the promoter is inducible, it may be a doxycycline inducible promoter (e.g., doxycycline inducible promoter 112 of FIGS. 1-4). In some embodiments, the doxycycline inducible promoter may be induced by a dose of doxycycline introduced to a plurality of cells, where the cells of the plurality cells include a nucleic acid (e.g., nucleic acid 110) including the doxycycline inducible promoter operatively coupled to the plasma membrane construct.

At step 512, the expressed plasma membrane polypeptide localizes to the plasma membrane of the cell at a certain rate 514. In some embodiments, the polypeptide is rhodopsin or a variant of rhodopsin. In some embodiments, the localization of the polypeptide to the plasma membrane of the cell is associated with alleviating or preventing certain diseases (e.g., by restoring some or all of the activity of the plasma membrane localized polypeptide). In some embodiments, the rate of localization 514 is indicative of the ability to alleviate or prevent the certain diseases. In some embodiments, a test agent is added to the plurality of cells. In those embodiments, the test agent may affect the ability of the plasma membrane polypeptide to localize to the plasma membrane. In some embodiments, the plurality of cells may be in a well. In some embodiments, the well may be part of a set of wells, for example as part of a of a multi-well plate or other container comprising a plurality of partitions. In some embodiments, each well of the set of wells may have a plurality of cells identical or similar to the plurality of cells described above. In some embodiments, a different test agent may be added to each well.

At step 522, the plasma membrane construct is cleaved by a protease at a rate of cleaving 524. In some embodiments, the plasma membrane construct is cleaved by a protease anchored to the plasma membrane (e.g., protease 140 of FIGS. 1-4). In some embodiments, the protease cleaves the plasma membrane construct at the linker. In some embodiments, the polypeptide stays localized to the plasma membrane. In some embodiments, a transcription factor of the plasma membrane construct is released into the cell. In this depicted embodiment, the rate of cleaving 524 is equal to the rate of binding multiplied by the coefficient “a”.

At step 532, the transcription factor binds to a nucleic acid comprising a promoter operatively coupled to a reporter gene (e.g., a UMI, luciferase, etc.) in the cell. In some embodiments, the nucleic acid includes the UMI and/or the luciferase gene. In some embodiments, the DNA binding domain binds to a promoter of the nucleic acid (e.g., plasma membrane proximity promoter 122). In some embodiments, the promoter is activated upon the binding of the transcription factor, which induces expression of the UMI and/or the luciferase gene, resulting in the UMI read count 536 (as determined by downstream sequencing) and/or rate of expression 534 of the luciferase gene (as determined by measurement of the luciferase enzymatic activity in the cell). In some embodiments, the UMI read count 536 is equal to the rate of cleaving 524 multiplied by a coefficient “b”. Thus, the UMI read count 536 is further equal to the rate of binding 514 multiplied by the coefficient “b” and the coefficient “a”. In some embodiments, the rate of expression 534 is equal to the rate of cleaving 524 multiple by a coefficient “c”. Thus, the rate of expression 534 is further equal to the rate of binding 514 multiplied by the coefficient “c” and the coefficient “a”. In some embodiments, the coefficient “b” is equal to the coefficient “c”. In some embodiments, the coefficient “b” is different from the coefficient “c”.

Thus, by determining the relationship of the UMI expression 536 and the rate of expression 534 at the end of the process using one or more next-generation sequencing techniques, the rate of localization of plasma membrane polypeptide 514 may be determined or inferred, indicating an ability to traffic the plasma membrane polypeptide to the plasma membrane of the cell, and thus, alleviate or prevent the certain diseases or conditions. Even further, with the use of multiple test agents with the set of wells, the rate of localization of plasma membrane polypeptide 514 associated with each test agent may be compared, and thus, the test agents may be compared based on their ability to assist in alleviating or preventing the certain diseases or conditions.

### Barcodes

Variable nucleotide sequences (unique molecular identifiers, also referred to herein as barcodes) that serve as an index can be included on any of the reporter genes described herein. Additionally, barcodes may be added in a separate library preparation reaction. The variable nucleotide sequences described herein can be used as a sample index in order to deconvolve results obtained from a sequencing reaction used herein.

Once the contents of the cells are released into their respective partitions by a lysis agent, the macromolecular components (e.g., macromolecular constituents of samples, such as RNA, DNA, or proteins) contained therein may be further processed within the partitions. In accordance with the methods and systems described herein, the macromolecular component contents of individual samples can be provided with unique identifiers such that, upon characterization of those macromolecular components they may be attributed as having been derived from the same sample or particles. The ability to attribute characteristics to individual samples or groups of samples is provided by the assignment of unique identifiers specifically to an individual sample or groups of samples. Unique identifiers, e.g., in the form of nucleic acid barcodes can be assigned or associated with individual samples or populations of samples, in order to tag or label the sample's macromolecular components (and as a result, its characteristics) with the unique identifiers. These unique identifiers can then be used to attribute the sample's components and characteristics to an individual sample or group of samples.

In some aspects, this is performed by co-partitioning the individual sample or groups of samples with the unique identifiers or barcodes comprising an unique molecular identifier sequence (UMI). In some aspects, the unique identifiers are provided in the form of nucleic acid molecules (e.g., oligonucleotides) that comprise nucleic acid barcode sequences that may be attached to or otherwise associated with the nucleic acid contents of individual sample, or to other components of the sample, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some embodiments, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some embodiments, the length of a barcode sequence may be about 6, 7, 8, 9, 10, 11, 12, 13, 14, 15, 16, 17, 18, 19, 20 nucleotides or longer. In some embodiments, the length of a barcode sequence may be at least about 6, 7, 8, 9, 10, 11, 12, 13, 14, 15, 16, 17, 18, 19, 20 nucleotides or longer. In some embodiments, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some embodiments, separated barcode subsequences can be from about 4 to about 16 nucleotides in length. In some embodiments, the barcode subsequence may be about 4, 5, 6, 7, 8, 9, 10, 11, 12, 13, 14, 15, 16 nucleotides or longer. In some embodiments, the barcode subsequence may be at least about 4, 5, 6, 7, 8, 9, 10, 11, 12, 13, 14, 15, 16 nucleotides or longer. In some embodiments, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned samples. These sequences include, e.g., targeted or random/universal amplification primer sequences for amplifying the genomic DNA from the individual samples within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more partitions, where one partition contains oligonucleotides, or microdispensing of oligonucleotides into partitions, e.g., partitions within microfluidic systems. In some embodiments, a primer comprises a barcode oligonucleotide. In some embodiments the primer sequence is a targeted primer sequence complementary to a sequence in the template nucleic acid molecule. In some embodiments, the first nucleic acid molecule further comprises one or more functional sequences and wherein the second nucleic acid molecule comprises the one or more functional sequences. In some embodiments, the one or more functional sequences are selected from the group consisting of an adapter sequence, an additional primer sequence, a primer annealing sequence, a sequencing primer sequence, a sequence configured to attach to a flow cell of a sequencer, and a unique molecular identifier sequence.

For example, the above-described barcoded nucleic acid molecules (e.g., barcoded oligonucleotides) are added to a sample. In some embodiments, a partition comprises barcoded oligonucleotides having the same barcode sequence. In some embodiments, a partition among a plurality of partitions comprises barcoded oligonucleotides having an identical barcode sequence, wherein each partition among within the plurality of partitions comprises a unique barcode sequence. In some embodiments, the population of barcoded oligonucleotides provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each barcoded oligonucleotide can be provided with large numbers of nucleic acid (e.g., oligonucleotide) molecules attached. In particular, the number of molecules of nucleic acid molecules including the barcode sequence on an individual barcoded oligonucleotide can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some embodiments at least about 1 billion nucleic acid molecules, or more. Nucleic acid molecules of a given barcoded oligonucleotide can include identical (or common) barcode sequences, different barcode sequences, or a combination of both. Nucleic acid molecules of a given barcoded oligonucleotide can include multiple sets of nucleic acid molecules. Nucleic acid molecules of a given set can include identical barcode sequences. The identical barcode sequences can be different from barcode sequences of nucleic acid molecules of another set.

Moreover, when the population of barcoded oligonucleotides is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some embodiments at least about 1 billion nucleic acid molecules.

In some embodiments, it may be desirable to incorporate multiple different barcodes within a given partition. For example, in some embodiments, a barcoded oligonucleotide within a partition can comprise (1) a common barcode sequence shared by all barcoded oligonucleotides within the partition and (2) a unique molecular identifier or additional barcode sequence that is different among each barcoded oligonucleotide. The common barcode sequences may provide greater assurance of identification in the subsequent processing, e.g., by providing a stronger address or attribution of the barcodes to a given partition, as a duplicate or independent confirmation of the output from a given partition.

In some embodiments, the barcoded oligonucleotides are attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein.

The nucleic acid molecules (e.g., oligonucleotides) can be releasable from the beads upon the application of a particular stimulus to the beads. In some embodiments, the stimulus may be a photo-stimulus, e.g., through cleavage of a photo-labile linkage that releases the nucleic acid molecules. In other embodiments, a thermal stimulus may be used, where elevation of the temperature of the beads environment will result in cleavage of a linkage or other release of the nucleic acid molecules form the beads. In still other embodiments, a chemical stimulus can be used that cleaves a linkage of the nucleic acid molecules to the beads, or otherwise results in release of the nucleic acid molecules from the beads. In one embodiment, such compositions include the polyacrylamide matrices described above for encapsulation of samples, and may be degraded for release of the attached nucleic acid molecules through exposure to a reducing agent, such as DTT.

A support can be contemplated for use in a method of the present disclosure may be, for example, a well, matrix, rod, container, or bead(s). A support may have any useful features and characteristics, such as any useful size, surface chemistry, fluidity, solidity, density, porosity, and composition. In some embodiments, a support is a surface of a well on a plate. In some embodiments, a support may be a bead such as a gel bead. A bead may be solid or semi-solid. Additional details of beads are provided elsewhere herein.

A support (e.g., a bead) may comprise an anchor sequence functionalized thereto (e.g., as described herein). An anchor sequence may be attached to the support via, for example, a disulfide linkage. An anchor sequence may comprise a partial read sequence and/or flow cell functional sequence. Such a sequence may permit sequencing of nucleic acid molecules attached to the sequence by a sequencer (e.g., an Illumina sequencer). Different anchor sequences may be useful for different sequencing applications. An anchor sequence may comprise, for example, a TruSeq or Nextera sequence. An anchor sequence may have any useful characteristics such as any useful length and nucleotide composition. For example, an anchor sequence may comprise 2, 3, 4, 5, 6, 7, 8, 9, 10, 11, 12, 13, 14, 15, 16, 17, 18, 19, 20, or more nucleotides. In some embodiments, an anchor sequence may comprise 15 nucleotides. Nucleotides of an anchor sequence may be naturally occurring or non-naturally occurring (e.g., as described herein). A bead may comprise a plurality of anchor sequences attached thereto. For example, a bead may comprise a plurality of first anchor sequences attached thereto. In some embodiments, a bead may comprise two or more different anchor sequences attached thereto. For example, a bead may comprise both a plurality of first anchor sequences (e.g., Nextera sequences) and a plurality of second anchor sequences (e.g., TruSeq sequences) attached thereto. For a bead comprising two or more different anchor sequences attached thereto, the sequence of each different anchor sequence may be distinguishable from the sequence of each other anchor sequence at an end distal to the bead. For example, the different anchor sequences may comprise one or more nucleotide differences in the 2, 3, 4, 5, 6, 7, 8, 9, 10, or more nucleotides furthest from the bead.

In some embodiments, multiple different barcode molecules (e.g., nucleic acid barcode molecules) may be generated on the same support (e.g., bead). For example, two different barcode molecules may be generated on the same support. Alternatively, three or more different barcode molecules may be generated on the same support. Different barcode molecules attached to the same support may comprise one or more different sequences. For example, different barcode molecules may comprise one or more different barcode sequences, and/or other sequences (e.g., starter sequences). In some embodiments, different barcode molecules attached to the same support may comprise the same barcode sequences. Different barcode molecules attached to the same support may comprise barcode sequences that are the same or different. Similarly, different barcode molecules may comprise unique molecular identifiers (UMIs) that are the same or different.

### Cells

Cells useful for the systems and methods described herein are generally those that are able to be easily rendered transgenic with one or more nucleic acids described herein. The system nucleic acid(s) encoding a regulatory element, an effector, and/or a reporter element can be transfected or transduced into suitable cell line using methods known in the art, such as calcium phosphate transfection, lipid based transfection (e.g., Lipofectamine™, Lipofectamine-2000™, Lipofectamine-3000™, or Fugene® HD), electroporation, or viral transduction. The cell can also be a population of cells of the same type grown to confluency or near confluency in an appropriate tissue culture vessel.

In certain embodiments, the cell used herein comprises a stable integration of either the nucleic acid encoding the regulatory element, the nucleic acid encoding the effector, the nucleic acid comprising the reporter element, or a combination thereof. Stable cell lines can be made from the cell described herein by using random integration of a linearized plasmid, virally or transposon directed integration, or directed integration, for example using site specific recombination between an AttP and an AttB site. In certain embodiments, either of the nucleic acids are integrated at a safe landing site such as the AAVS1 site.

In some embodiments, the cell described herein comprises the nucleic acid stably integrated into the genome of the cell. In some embodiments, the cell described herein comprises the nucleic acid encoding the regulatory element stably integrated into the genome of the cell. In some embodiments, the cell described herein comprises the nucleic acid encoding at least one effector described herein stably integrated into the genome of the cell. In some embodiments, the cell comprises stably integrated nucleic acid encoding a regulatory element for modulating expression of the effector. In some cases, the cell comprises stably integrated nucleic acid encoding a regulatory element for upregulating the effector described herein. In some cases, the cell comprises stably integrated nucleic acid encoding a regulatory element for upregulating ADCY6. In some cases, the cell comprises stably integrated nucleic acid encoding ADCY6. In some cases, the cell comprises stably integrated nucleic acid encoding a regulatory element for downregulating the effector described herein. In some cases, the cell comprises stably integrated nucleic acid encoding a regulatory element for downregulating ADCY3.

In certain embodiments, the cell or cell population used in the system is a eukaryotic cell. In certain embodiments, the cell or cell population is a mammalian cell. In certain embodiments, the cell or cell population is a human cell. In certain embodiments, the cell or cell population is SH-SY5Y, Human neuroblastoma; Hep G2, Human Caucasian hepatocyte carcinoma; 293 (also known as HEK 293), Human Embryo Kidney; RAW 264.7, Mouse monocyte macrophage; HeLa, Human cervix epitheloid carcinoma; MRC-5 (PD 19), Human fetal lung; A2780, Human ovarian carcinoma; CACO-2, Human Caucasian colon adenocarcinoma; THP 1, Human monocytic leukemia; A549, Human Caucasian lung carcinoma; MRC-5 (PD 30), Human fetal lung; MCF7, Human Caucasian breast adenocarcinoma; SNL 76/7, Mouse SIM strain embryonic fibroblast; C2C12, Mouse C3H muscle myoblast; Jurkat E6.1, Human leukemic T cell lymphoblast; U937, Human Caucasian histiocytic lymphoma; L929, Mouse C3H/An connective tissue; 3T3 L1, Mouse Embryo; HL60, Human Caucasian promyelocytic leukaemia; PC-12, Rat adrenal phaeochromocytoma; HT29, Human Caucasian colon adenocarcinoma; OE33, Human Caucasian oesophageal carcinoma; OE19, Human Caucasian oesophageal carcinoma; NIH 3T3, Mouse Swiss NIH embryo; MDA-MB-231, Human Caucasian breast adenocarcinoma; K562, Human Caucasian chronic myelogenous leukemia; U-87 MG, Human glioblastoma astrocytoma; MRC-5 (PD 25), Human fetal lung; A2780cis, Human ovarian carcinoma; B9, Mouse B cell hybridoma; CHO-K1, U2OS, Hamster Chinese ovary; MDCK, Canine Cocker Spaniel kidney; 1321N1, Human brain astrocytoma; A431, Human squamous carcinoma; ATDC5, Mouse 129 teratocarcinoma AT805 derived; RCC4 PLUS VECTOR ALONE, Renal cell carcinoma cell line RCC4 stably transfected with an empty expression vector, pcDNA3, conferring neomycin resistance; HUVEC (S200-05n), Human Pre-screened Umbilical Vein Endothelial Cells (HUVEC); neonatal; Vero, Monkey African Green kidney; RCC4 PLUS VHL, Renal cell carcinoma cell line RCC4 stably transfected with pcDNA3-VHL; Fao, Rat hepatoma; J774A.1, Mouse BALB/c monocyte macrophage; MC3T3-E1, Mouse C57BL/6 calvaria; J774.2, Mouse BALB/c monocyte macrophage; PNT1A, Human post pubertal prostate normal, immortalised with SV40; U-2 OS, Human Osteosarcoma; HCT 116, Human colon carcinoma; MA104, Monkey African Green kidney; BEAS-2B, Human bronchial epithelium, normal; NB2-11, Rat lymphoma; BHK 21 (clone 13), Hamster Syrian kidney; NS0, Mouse myeloma; Neuro 2a, Mouse Albino neuroblastoma; SP2/0-Ag14, Mouse×Mouse myeloma, non-producing; T47D, Human breast tumor; 1301, Human T-cell leukemia; MDCK-II, Canine Cocker Spaniel Kidney; PNT2, Human prostate normal, immortalized with SV40; PC-3, Human Caucasian prostate adenocarcinoma; TF1, Human erythroleukaemia; COS-7, Monkey African green kidney, SV40 transformed; MDCK, Canine Cocker Spaniel kidney; HUVEC (200-05n), Human Umbilical Vein Endothelial Cells (HUVEC); neonatal; NCI-H322, Human Caucasian bronchioalveolar carcinoma; SK.N.SH, Human Caucasian neuroblastoma; LNCaP.FGC, Human Caucasian prostate carcinoma; OE21, Human Caucasian oesophageal squamous cell carcinoma; PSN1, Human pancreatic adenocarcinoma; ISHIKAWA, Human Asian endometrial adenocarcinoma; MFE-280, Human Caucasian endometrial adenocarcinoma; MG-63, Human osteosarcoma; RK 13, Rabbit kidney, BVDV negative; EoL-1 cell, Human eosinophilic leukemia; VCaP, Human Prostate Cancer Metastasis; tsA201, Human embryonal kidney, SV40 transformed; CHO, Hamster Chinese ovary; HT 1080, Human fibrosarcoma; PANC-1, Human Caucasian pancreas; Saos-2, Human primary osteogenic sarcoma; Fibroblast Growth Medium (116K-500), Fibroblast Growth Medium Kit; ND7/23, Mouse neuroblastoma×Rat neuron hybrid; SK-OV-3, Human Caucasian ovary adenocarcinoma; COV434, Human ovarian granulosa tumor; Hep 3B, Human hepatocyte carcinoma; Vero (WHO), Monkey African Green kidney; Nthy-ori 3-1, Human thyroid follicular epithelial; U373 MG (Uppsala), Human glioblastoma astrocytoma; A375, Human malignant melanoma; AGS, Human Caucasian gastric adenocarcinoma; CAKI 2, Human Caucasian kidney carcinoma; COLO 205, Human Caucasian colon adenocarcinoma; COR-L23, Human Caucasian lung large cell carcinoma; IMR 32, Human Caucasian neuroblastoma; QT 35, Quail Japanese fibrosarcoma; WI 38, Human Caucasian fetal lung; HMVII, Human vaginal malignant melanoma; HT55, Human colon carcinoma; TK6, Human lymphoblast, thymidine kinase heterozygote; SP2/0-AG14 (AC-FREE), Mouse×mouse hybridoma non-secreting, serum-free, animal component (AC) free; AR42J, or Rat exocrine pancreatic tumor, or any combination thereof.

### Methods for Measuring Plasma Membrane Proximity Reporter Activities Through Use of Unique Molecular Identifiers

One example of a method for measuring plasma membrane activities after exposure to a test agent through the use of a unique molecular identifier is described below.

In the non-limiting example, a plurality of cells are incubated in one well of a multi-well plate. The plurality of cells are transfected with at least one nucleic acid described here (e.g., nucleic acid 110, nucleic acid 120, and/or nucleic acid 130 of FIGS. 1-4). In some embodiments, at least one nucleic acid encodes a regulatory element described herein (e.g., any combination of doxycycline inducible promoter 112, plasma membrane polypeptide 114, plasma membrane proximity promoter 122, luciferase gene 124, UMI 126, promoter 132, and/or protease 134). In some embodiments, at least one nucleic acid encodes a reporter gene described herein (e.g., including luciferase gene 124 and UMI 126, or including unique molecular identifier 126 alone). In some embodiments, at least one nucleic acid encodes a doxycycline inducible promoter in operable combination with the plasma membrane construct. In some embodiments, at least one nucleic acid encodes a promoter in operable combination with a protease. In this example, the plurality of cells are introduced to doxycycline, which may increase activation of the doxycycline inducible promoter in the plurality of cells, which may lead to an increase in expression of the plasma membrane polypeptide (e.g., rhodopsin), a linker with a cleavage site, and a transcription factor.

The plurality of cells may then be exposed to a test agent. The exposure to the dose of test agent may improve the rate of trafficking of the plasma membrane polypeptide to the plasma membrane of the plurality of cells. The plasma membrane polypeptide may localize to the plasma membrane of the plurality of cells. The plasma membrane construct then may be cleaved at the cleavage site of the linker, leaving the polypeptide remaining localized at the plasma membrane while the transcription factor is released.

The transcription factor may bind to a plasma membrane proximity promoter of a nucleic acid once released. The binding of the transcription factor to the plasma membrane proximity promoter may induce expression of a unique molecular identifier. The expression of the unique molecular identifier may then be used to evaluate the ability of the cells in alleviating certain diseases or conditions (e.g., retinitis pigmentosa, Alzheimer's disease, Parkinson's Disease, Huntington disease, Cystic Fibrosis, Alpha-1 Anti-trypsin Disorder, or Amyotrophic Lateral Sclerosis) based on the ability of the plasma membrane construct (e.g., at the polypeptide) to bind the plasma membrane of the cell.

Additionally, the plurality of cells being exposed to the test agent may additionally allow the test agent to interact with the components of the cell, as described above. The test agent may, in particular, interact with the polypeptide or components of the cell important for the biosynthesis and proper trafficking of the polypeptide, thus, increasing or decreasing the polypeptide's ability to localize at the plasma membrane of the cell. Thus, as a result of the change (e.g., increase or decrease) in the polypeptide's ability to localize at the plasma membrane of the cell, the transcription factor may or may not be released (e.g., because the linker may or may not be cleaved), which may directly affect (e.g., increase or decrease) the expression of the unique molecular identifier. Thus, by observing the change in the expression of the unique molecular identifier as a result of the exposure to the test agent, the effect of the test agent on the ability of the polypeptide to localize at the plasma membrane of the cell, and thus the effect of the test agent on a cell's ability to alleviate certain diseases or conditions, may also be observed.

### Next Generation Sequencing

As described in the methods disclosed herein, the sequencing of nucleic acid molecules is used and is useful for the detection of biological effect by a test agent against a cell comprising a cell based assay. Generally, sequencing refers to methods and technologies for determining the sequence of nucleotide bases in one or more polynucleotides. The polynucleotides can be, for example, deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina, Pacific Biosciences, Oxford Nanopore, or Life Technologies (Ion Torrent). Such devices may provide a plurality of raw genetic data corresponding to the genetic information of a subject (e.g., human), as generated by the device from a sample provided by the subject. In some situations, systems and methods provided herein may be used with proteomic information. Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

Next generation sequencing includes many technologies capable of generating large amounts of sequence information and excluding Sanger sequencing or Maxam-Gilbert sequencing. Generally, next generation sequencing encompasses single molecule real-time sequencing, sequencing-by-synthesis, ion semiconductor sequencing and the like. Exemplary next-generation sequencing machines may comprise the MiniSeq, the iSeq100, the NextSeq 1000, the NextSeq 2000, the NovaSeq 6000, the NextSeq 550 series and the like from Illumina, Inc; Ion Torrent machines from Thermo Fisher Scientific; or the Sequel systems from Pacific Biosciences.

Next generation sequencing machines used with the method herein can generate at least 1, 5, 10, 15, 25, 50, 75, 100, 200, 300 gigabases of data or more in a 24 hour period from a single machine.

Next generation sequencing machines used with the method herein can generate at least 1, 1, 4, 10, 15, 25, 50, 75, 100, 200, 300, 500, or 1,000 million sequence reads of data or more in a 24 hour period from a single machine.

Also included is a computer program, computing device, or analysis platform/system to receive and analyze sequencing data, and output one or more reports that can be transmitted or accessed electronically via a server, an analysis portal, or by e-mail. The computing device or analysis platform can operate according to the algorithms and methods described herein.

The nucleic acids of the present disclosure are compatible with many vectors common in the art. Non-limiting examples of vectors include genomic integrated vectors, episomal vectors, plasmids, viral vectors, cosmids, bacterial artificial chromosomes, and yeast artificial chromosomes. Non-limiting examples of viral vectors compatible with the nucleic acids of the present disclosure include vectors derived from lentiviruses, retroviruses, adenoviruses, and adeno-associated viruses. In certain embodiments, the nucleic acids of the present disclosure are present on vectors comprising sequences that direct site specific integration into a defined location or a restricted set of sites in the genome (e.g., AttP-AttB recombination).

In certain embodiments, the system described herein is incorporated into a single vector. In certain embodiments, the single vector is transfected into a cell transiently. In certain embodiments, the single vector is transfected into a cell stably.

In certain embodiments, the system is divided across two vectors. In certain embodiments, a first vector comprises a first regulatory element and a first effector, while a second vector comprises a second regulatory element for modulating the expression of a second effector. In certain embodiments, the first vector and the second vector are transiently transfected into a cell. In certain embodiments, the first vector and the second vector are stably transfected into a cell. In certain embodiments, the first vector is transfected into a cell stably and the second vector is transfected into a cell transiently. In certain embodiments, the first vector is transfected into a cell transiently and the second vector is transfected into a cell stably. In some embodiments, a separate vector comprising the reporter can be transfected into the cell. In some embodiments, the cell transfected with the first or second vector already comprises the reporter.

Vectors comprising the systems described herein or portions thereof may be constructed using many well-known molecular biology techniques. Detailed protocols for numerous such procedures, including amplification, cloning, mutagenesis, transformation, and the like, are described in, e.g., in Ausubel et al. Current Protocols in Molecular Biology (supplemented through 2012) John Wiley & Sons, New York 10 (“Ausubel”); Sambrook et al. Molecular Cloning-A Laboratory Manual (4th Ed.), Vol. 1-3, Cold Spring Harbor Laboratory, Cold Spring Harbor, New York, 2012 (“Sambrook”); and Abelson et al. Guide to Molecular Cloning Techniques (Methods in Enzymology) volume 152 Academic Press, Inc., San Diego, CA (“Abelson”).

### Amenability Assays

Described herein are methods of determining amenability for a test agent to rescue defective plasma membrane trafficking of a variant plasma membrane protein of interest. Provided herein are methods comprising: (a) expressing a recombinant form of the variant plasma membrane protein in a host cell and contacting the host cell with the test agent; (b) measuring trafficking of the variant plasma membrane protein to the plasma membrane of the host cell using any one of the systems provided herein; (c) comparing the trafficking determined in (b) to the trafficking in the host cell when it is not contacted with the test agent, and (d) determining that a patient afflicted with or predisposed to a disease associated with the variant plasma membrane protein is a candidate for treatment with the test agent if the trafficking of the plasma membrane protein is increased in the host cell contacted with the test agent when compared to trafficking in the host cell not contacted with the test agent. In some embodiments, step (d) comprises determining that the patient is a candidate for treatment with the test agent if, in step (c), there is at least a 1.3 to 40 fold increase in trafficking in the host cell contacted with the test agent when compared to trafficking in the host cell not contacted with the test agent. In some embodiments, the increase in trafficking in the host cell contacted with the test agent can be at least about 1.1, 1.2, 1.3, 1.4, 1.5, 1.6, 1.7, 1.8, 1.9, 2.0, 2.2, 2.4, 2.6, 2.8, 3.0, 3.2, 3.4, 3.6, 3.8, 4.0, 4.2, 4.4, 4.6, 4.8, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 25.0, 30.0, 35.0, 40.0, 45.0, 50.0, 55.0, 60.0, 65.0, 70.0, 75.0, 80.0, 85.0, 90.0, 95.0, or 100.0 fold greater when compared to trafficking in the host cell not contacted with the test agent. In some embodiments, step (d) comprises determining that the patient is a candidate for treatment with the test agent if the trafficking in the host cell is at least 2% to about 100% of a non-mutant plasma membrane protein. In some embodiments, the trafficking in the host cell can be at least about 1%, 2%, 3%, 4%, 5%, 6%, 7%, 8%, 9%, 10%, 11%, 12%, 13%, 14%, 15%, 16%, 17%, 18%, 19%, 20%, 22%, 23%, 24%, 26%, 28%, 30%, 32%, 34%, 36%, 38%, 40%, 42%, 44%, 46%, 48%, 50%, 55%, 60%, 65%, 70%, 75%, 80%, 85%, 90%, 95%, or 100% of a non-mutant plasma membrane protein.

In some embodiments, the variant plasma membrane protein or a gene encoding the variant plasma membrane protein has been identified from the patient afflicted with or predisposed to a disease associated with the variant plasma membrane protein.

In some embodiments, the test agent is a pharmacological corrector.

### Kits

In some embodiments, the kit comprises the system described herein, which can be used to perform the methods described herein. Kits comprise an assemblage of materials or compositions, including at least one of the composition of the system. In other embodiments, the kits contains all of the compositions necessary and/or sufficient to perform the methods described herein, including all controls and directions.

In some cases, instructions for use can be included in the kit. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### EXAMPLES

Specialized processes that could inform on the performance of the workflows described herein was instituted. These processes comprised adding doxycycline and/or a dose of one or more test agents to a plurality of cells (e.g., the plurality of cells 100 of FIGS. 1-4) as well as measuring expression of certain polypeptides (e.g., polypeptide 152 of FIGS. 3-4) and/or unique molecular identifiers (e.g., unique molecular identifier 126 of FIGS. 1-4). Sequencing readouts of these polypeptides and unique molecular identifiers are made at the end of the multiplexed assay, and results provide key insights on how the one or more components introduced to the populations of cells affects the ability of the polypeptide to localize at the plasma membrane of a cell. Specific examples provided herein include, but are not limited to, assays to identify pharmacological correctors (e.g., chaperones) capable of restoring plasma membrane expression of particular mutant polypeptides that do not properly traffic to the plasma membrane, as well as amenability assays to identify mutations of plasma membrane polypeptides that can be treated using a particular pharmacological corrector.

### Example 1: Evaluating Rhodopsin Expression Using a Unique Molecular Identifier Output

An HTS assay is run with cells in wells of a 384 well plate, with each well containing about 20,000 cells. The cells include a first nucleic acid sequence encoding a plasma membrane construct comprising a doxycycline inducible promoter operatively coupled to a nucleic acid sequence encoding a plasma membrane polypeptide, in this example, a Rhodopsin polypeptide, and a second nucleic acid sequence comprising a reporter construct promoter (i.e., a plasma membrane proximity promoter) operatively coupled to a luciferase gene and a unique molecular identifier.

A dose of doxycycline is added to each well of the 384 well plate to induce expression of the rhodopsin polypeptide. Expression of the rhodopsin polypeptide allows for the rhodopsin polypeptide to be transported to the plasma membrane of the cells.

After the rhodopsin polypeptide localizes to the plasma membrane of the cell, a linker of the plasma membrane construct including the plasma membrane polypeptide is cleaved at a cleavage site by a protease anchored to the plasma membrane. A transcription factor of the plasma membrane construct, which includes a DNA binding domain and a transcriptional activator, is released into the cells as a result.

The DNA binding domain of the transcription factor activates the promoter of the second nucleic acid to induce expression of the luciferase gene and the unique molecular identifier, where the expression of the unique molecular identifier is related to the rate at which the rhodopsin polypeptide localize at the plasma membrane of the cells.

The output of a luminometer after addition of a luciferase agent is determined, indicating the expression of the unique molecular identifier.

### Example 2: Evaluating Rhodopsin Expression Using Unique Molecular Identifier Output in Response to a Test Agent

To begin, a first HTS assay according to example 1 is first run.

Then, a second HTS assay is run with cells in wells of a 384 well plate, with each well containing about 20,000 cells. The cells include a first nucleic acid sequence encoding a plasma membrane construct comprising a doxycycline inducible promoter operatively coupled to a nucleic acid sequence encoding a plasma membrane polypeptide, in this example a Rhodopsin polypeptide, and a second nucleic acid sequence comprising a reporter construct promoter (i.e., a plasma membrane proximity promoter) operatively coupled to a luciferase gene and a unique molecular identifier. A dose of doxycycline is added to each well of the 384 well plate to induce expression of the rhodopsin polypeptide. Expression of the rhodopsin polypeptide allows for the rhodopsin polypeptide to be transported to the plasma membrane of the cells.

A dose of test agent is then added to each well of the 384 well plate in order to evaluate the effect of the test agent on the expression and related trafficking of the rhodopsin polypeptide to the plasma membrane of the cell.

After the rhodopsin polypeptide is localized to the plasma membrane, the plasma membrane construct including the expressed rhodopsin polypeptide, a linker, and a transcription factor is cleaved at the linker. More specifically, the linker is cleaved at a cleavage site by a protease anchored to the plasma membrane of the cell. The rhodopsin polypeptide then remains at the plasma membrane while the transcription factor, which includes a DNA-binding domain and a transcriptional activator, is released.

The DNA binding domain of the transcription factor activates the promoter of the second nucleic acid to induce expression of the luciferase gene and the unique molecular identifier, where the expression of the unique molecular identifier is related to the rate at which the rhodopsin polypeptide localize at the plasma membrane of the cells.

The output of a luminometer after addition of a luciferase agent is determined, indicating the expression of the unique molecular identifier. The output of the luminometer associated with the wells of this example are then compared to the output of the luminometer associated with the wells of example 1. The expression of the unique molecular identifier for the wells of the second assay is the compared to the expression of the unique molecular identifier for the wells of the first assay in order to determine if the test agent caused an increase in readouts of the unique molecular identifier, which consequently allowed for evaluation of the trafficking of the rhodopsin polypeptide to the plasma membrane for the second assay as compared to the first assay, thus indicating whether the added test agent increased or decreased trafficking of the rhodopsin polypeptide.

### Example 3: Amenability Assay

The systems and methods described herein can also be used to perform an amenability assay to evaluate trafficking consequences of every single possible amino acid change in a particular plasma membrane polypeptide. The trafficking consequences of every single possible amino acid change in a particular plasma membrane polypeptide, or at least of each pathogenic mutation identified in patients having certain conditions or diseases (e.g., retinitis pigmentosa, GLP-1R downregulation, cystic fibrosis, etc.), can also be assessed in the presence of a test agent to determine whether treatment with the test agent is likely to be effective at correcting plasma membrane trafficking of a pathogenic mutant polypeptide.

DNA constructs encoding (i) a plasma membrane polypeptide construct comprising a variant of a plasma membrane polypeptide, (ii) a plasma membrane anchored protease, and (iii) a reporter with a variant-specific barcode can be stably integrated into HEK293T cells at single copy. A cell line can be made for each variant of the plasma membrane polypeptide, each cell line having a unique molecular identifier that can be associated with each variant. Cells from each cell line can be pooled and plated in 15-cm well plate in DMEM 10% FBS. Expression of the plasma membrane polypeptide constructs can be driven by a doxycycline-inducible promoter. Expression of the plasma membrane anchored protease can be driven by a constitutive promoter. Test agents and/or doxycycline can be applied to cells immediately after plating. The cells can incubate for 24 hours at 37 °C and 5% CO₂ in a cell culture incubator. Cells can be lysed after incubating. RNA barcodes can be selectively reverse transcribed (primer upstream of barcode) to generate cDNA, which can be amplified to prepare NGS libraries. Barcodes can be sequenced, and barcode counts can be modeled with a negative binomial generalized linear model to determine variant effects relative to wild-type. A barcode associated with a variant plasma membrane polypeptide will be sequenced if the linker between the transcription factor and the variant plasma membrane polypeptide is cleaved by the plasma membrane anchored protease.

Using the methods and systems provided herein, the trafficking consequence of every single possible amino acid change in, e.g., the rhodopsin or GLP-1R protein, or any other plasma membrane targeted protein, can be assessed, both in the presence or absence of a test agent (e.g., a chaperone or pharmacological corrector).

### Example 4: Optimizing the Protease Cleavage Site of the Linker

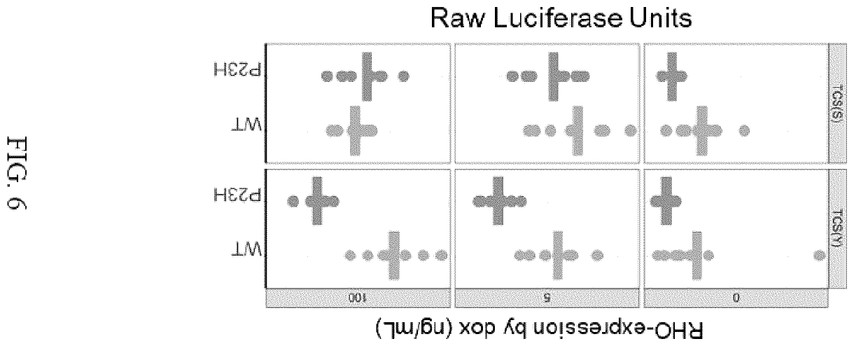

The plasma membrane construct linker between the plasma membrane polypeptide and a transcription factor can be cleaved at a cleavage site by a protease anchored to the plasma membrane of the cell. A variety of proteases are useful for the systems described herein. Experiments were conducted to determine the optimal cleavage site for the Tobacco Etch Virus (“TEV”)-S219V variant protease, which can cleave the sequence ENLYFQ(X) (SEQ ID NO: 22). FIG. 6 depicts an optimized cleavage site sequence for cleavage by TEV, showing a comparison between serine and tyrosine substituted within the (X) amino acid. In this example, rhodopsin was the plasma membrane construct. RHO-WT is wildtype rhodopsin, and RHO-P23H is a mutant that expresses poorly at the plasma membrane. Rhodopsin expression was induced by doxycycline. At low expression, the difference between TCS(S) (i.e., ENLYFQ/S) and TCS (Y) (i.e., ENLYFQ/Y) is minimal. Unexpectedly, at higher expressions, TCS (Y) appears to be the more optimal protease cleavage site for TEV, and the reporter is better able to distinguish between RHO-WT vs. RHO-P23H expression at the plasma membrane.

### Example 5: Optimizing the Plasma Membrane Protease Anchor and Promotor

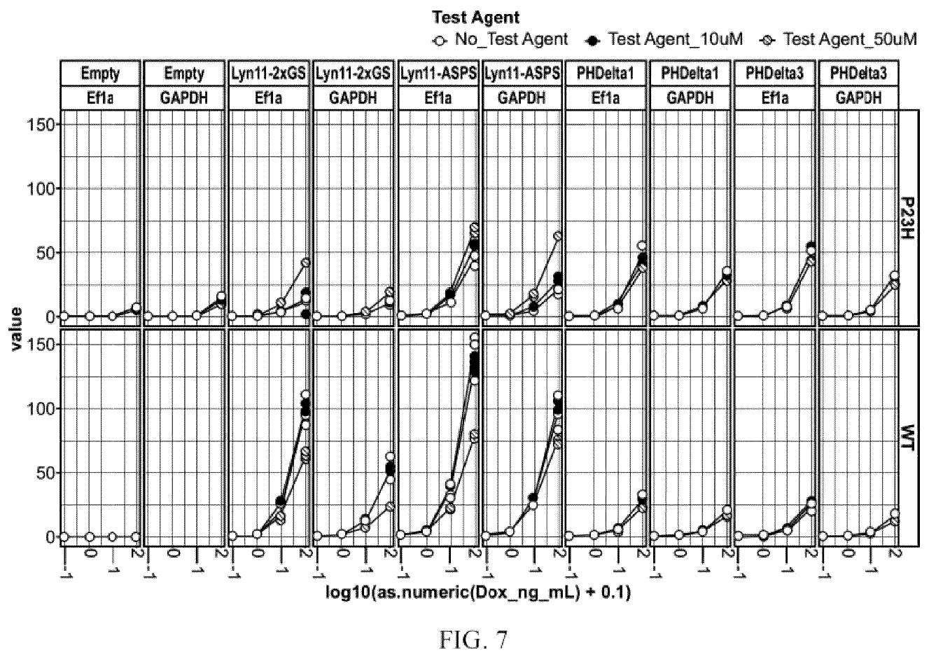

The plasma membrane anchored protease can be encoded by an exogenous nucleic acid. A promoter is operatively coupled to a nucleic acid sequence encoding a plasma membrane anchor (e.g., Lyn11, PHDelta1, PHDelta3) and a protease (e.g., TEV) joined by a linker (e.g., ASPSNPGASNGS (“ASPS”; SEQ ID NO: 39) or GGGGSGGGGS (“2×GS”; SEQ ID NO: 40)). When the promoter is activated, the plasma membrane anchor and the protease can be expressed. When targeted or localized to the plasma membrane, the protease may be capable of cleaving a plasma membrane construct linker that joins the plasma membrane polypeptide to the transcription factor at a cleavage site. Experiments were conducted to determine the optimal protease anchor and/or promoter. FIG. 7 depicts a comparison between using GAPDH and efla for the constitutive promoter driving plasma membrane-anchored TEV. Different membrane anchors were also tested, indicating the effectiveness of Lyn11 vs. PHDelta variants for detecting test agent rescue of RHO-P23H plasma membrane trafficking. In this examples, a 2×GS linker was used unless ASPS is specified. Unexpectedly, the Lyn11-ASPS anchor-linker pair worked the best at detecting test agent rescue of RHO-P23H plasma membrane trafficking. Lyn11-2×GS was shown to work better when the constitutive efla promoter was used as compared to GAPDH.

### Example 6: Optimizing the DNA Binding Domain (DBD)

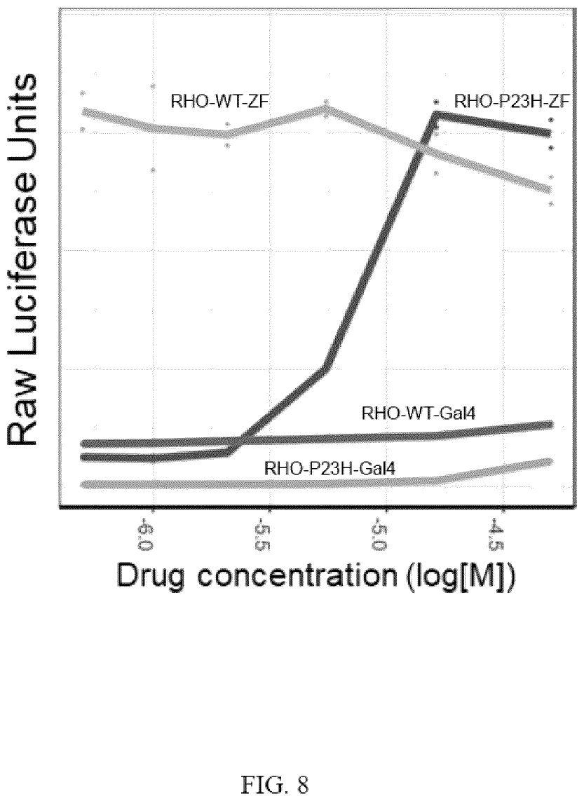

A transcription factor comprising a DBD and transactivator can be linked to the plasma membrane polypeptide. When the plasma membrane anchored protease cleaves the linker, the transcription factor can bind to a reporter construct promoter and drive expression of a reporter construct. Experiments were conducted to determine the optimal DBD for detecting rescue of plasma membrane construct trafficking. FIG. 8 depicts a comparison of Gal4 and ZF (i.e., RARR) DBD for detecting rescue of RHO-P23H trafficking by a test agent. Unexpectedly, ZF worked the best to detect rescue of RHO-P23H at higher concentrations of the test agent. The DBD responsive promoter can be further optimized for use with ZF, e.g., the DBD responsive promoter can be made to have up to 12 ZF binding sites.

### Example 7: Luciferase Protocol

DNA constructs encoding a RHO-TF, PM-TEV and luciferase reporter can be stably integrated into HEK293T cells. RHO-TEV is a rhodopsin tethered transcription factor. PM-TEV is a plasma membrane anchored TEV protease capable of cleaving the linker between the rhodopsin and the transcription factor. The transcription factor binds a promoter driving expression of the luciferase reporter. Cells can be plated in 384-well plates at 20,000 cells/well in DMEM 10% FBS. Expression of RHO-TF can be driven by a doxycycline-inducible promoter. Expression of PM-TEV can be driven by a constitutive promoter. When doxycycline is applied, test agents or other testing conditions can be added to determine an effect on plasma membrane trafficking of RHO-TF. The cells can incubate for 24 hours at 37 C and 5% CO₂ in a cell culture incubator. A luciferase readout can be obtained on a luminometer according to the Promega Bright-Glo™ assay system.

### Example 8: Broad Target Scanning (BTS) Protocol

DNA constructs encoding the RHO-TF, PM-TEV and a reporter can be stably integrated into HEK293T cells. In this example, the reporter is a variant-specific barcode. Cell-library can be generated by pooling the integrated cells. Cells can be plated in 384-well plates at 20,000 cells/well in DMEM 10% FBS. Expression of RHO-TF can be driven by a doxycycline-inducible promoter. Expression of PM-TEV can be driven by a constitutive promoter. Test agents and/or doxycycline can be applied to cells immediately after plating. The cells can incubate for 24 hours at 37 C and 5% CO2 in a cell culture incubator. Cells can be lysed after incubating. RNA barcodes can be selectively reverse transcribed (primer upstream of barcode) to generate cDNA, which can be amplified to prepare NGS libraries. Barcodes can be sequenced, and barcode counts can be modeled with a negative binomial generalized linear model to determine variant and effects of test compounds.

### Example 9: Multiplexable Trafficking Assay Via Broad Target Scanning (BTS)

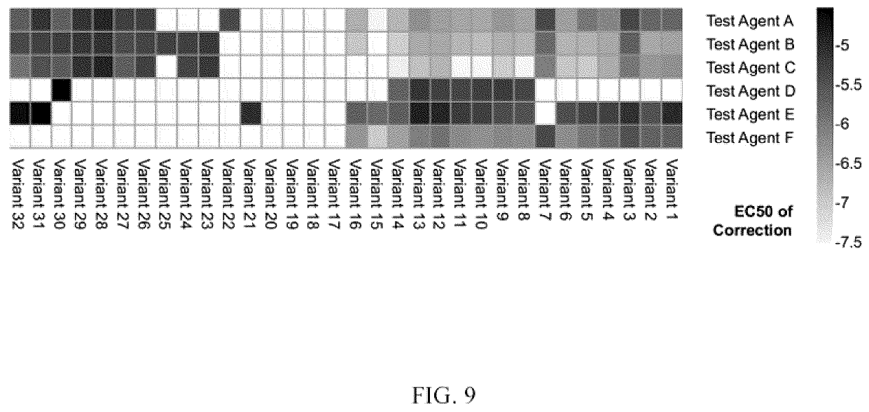

A 384-well plate can be set up to measure the trafficking consequences of 50-100 variants simultaneously. Dose response curves for plasma trafficking rescue can be collected against dozens of variants simultaneously at the equivalence of 264 dose-response curves (17,000 data points) collected from a single 384-well plate. FIG. 9 depicts specificity of 6 different test agents across 32 different pathogenic autosomal dominant retinitis pigmentosa (ADRP) variants using BTS. The greyscale reflects the level of potency against that mutation.

### Example 10: Deep Mutational Scanning (DMS) Protocol

DNA constructs encoding the RHO-TF, PM-TEV and reporter with a variant-specific barcode can be stably integrated in pool format into HEK293T cells at single copy. Cells can be plated in 15-cm well plate in DMEM 10% FBS. Expression of RHO-TF can be driven by a doxycycline-inducible promoter. Expression of PM-TEV can be driven by a constitutive promoter. Test agents and/or doxycycline can be applied to cells immediately after plating. The cells can incubate for 24 hours at 37 C and 5% CO₂ in a cell culture incubator. Cells can be lysed after incubating. RNA barcodes can be selectively reverse transcribed (primer upstream of barcode) to generate cDNA, which can be amplified to prepare NGS libraries. Barcodes can be sequenced, and barcode counts can be modeled with a negative binomial generalized linear model to determine variant effects relative to wild-type.

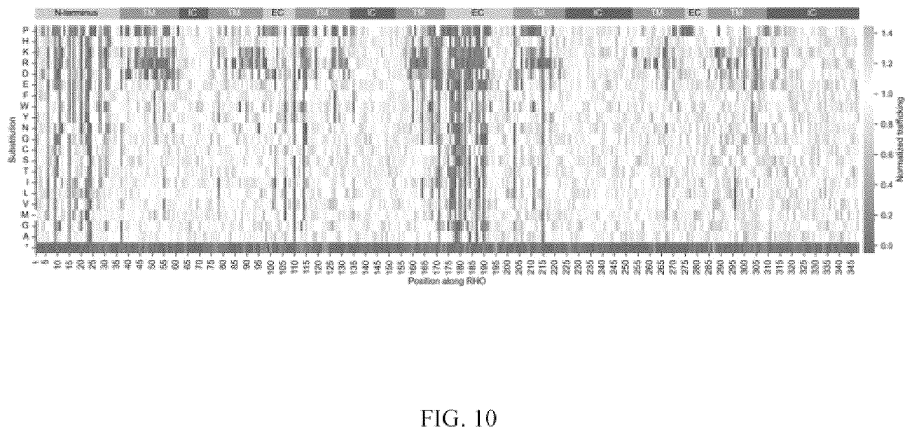

FIG. 10 depicts how the multiplexable trafficking assay was used to measure the trafficking consequence of every single possible amino acid change in the rhodopsin protein via DMS. Trafficking score is normalized to wildtype (“WT”) trafficking at a value of 1.0.

While preferred embodiments of the systems and methods have been shown and described herein, it will be obvious to those skilled in the art that such embodiments are provided by way of example only. It is not intended that the systems and methods be limited by the specific examples provided within the specification. While the systems and methods have been described with reference to the aforementioned specification, the descriptions and illustrations of the embodiments herein are not meant to be construed in a limiting sense. Numerous variations, changes, and substitutions will now occur to those skilled in the art without departing from the present disclosure. Furthermore, it shall be understood that all aspects of the systems and methods are not limited to the specific depictions, configurations or relative proportions set forth herein which depend upon a variety of conditions and variables. It should be understood that various alternatives to the embodiments of the systems and methods described herein may be employed in practicing the present disclosure. It is therefore contemplated that the systems and methods described herein shall also cover any such alternatives, modifications, variations or equivalents. It is intended that the following claims define the scope of the systems and methods described herein and that the systems and methods described herein are within the scope of these claims and their equivalents be covered thereby.

### NUMBERED EMBODIMENTS

1. A system comprising a eukaryotic cell, wherein the eukaryotic cell comprises: (a) a plasma membrane construct (PMC) comprising a plasma membrane polypeptide coupled to a transcription factor by a PMC linker, wherein the linker comprises a protease cleavage site; and (b) a plasma membrane anchored protease; wherein the plasma membrane anchored protease is capable of cleaving the linker.
2. The system of embodiment 1, further comprising a reporter construct comprising a promoter and a reporter gene.
3. The system of embodiment 2, wherein the promoter is bound by the transcription factor upon cleavage of the linker.
4. The system of any one of embodiments 1 to 3, wherein the plasma membrane construct is encoded by an exogenous nucleic acid.
5. The system of any one of embodiments 2 to 4, wherein the reporter construct is encoded by an exogenous nucleic acid.
6. The system of any one of embodiments 2 to 5, wherein the reporter gene comprises a unique molecular identifier.
7. The system of any one of embodiments 2 to 6, wherein the reporter gene comprises a fluorescent protein or a luciferase protein.
8. The system of any one of embodiments 2 to 7, wherein the reporter gene comprises a fluorescent protein and a unique molecular identifier or a luciferase protein and a unique molecular identifier.
9. The system of any one of embodiments 1 to 8, wherein the plasma membrane anchored protease is integral to the plasma membrane of the eukaryotic cell.
10. The system of any one of embodiments 1 to 9, wherein the plasma membrane anchored protease comprises a membrane tethered protease.
11. The system of embodiment 10, wherein the membrane tethered protease comprises a Pleckstrin Homology Domain, platelet-derived growth factor receptor, or Lyn anchor domain.
12. The system of any one of embodiments 1 to 12, wherein the transcription factor comprises a DNA binding domain and a transcriptional activation domain.
13. The system of embodiment 12, wherein the DNA binding domain comprises a Gal4, PPR1, Lac9, zinc finger, or LexA DNA binding domain.
14. The system of embodiment 12 or 13, wherein the transcriptional activation domain comprises a VP64, VPr, p65, Rta, or VP16 activation domain.
15. The system of any one of embodiments 1 to 14, wherein the linker comprises a flexible amino acid linker.
16. The system of any one of embodiments 1 to 15, wherein the linker has a length of about 2 to about 31 amino acids.
17. The system of any one of embodiments 1 to 16, wherein the plasma membrane anchored protease comprises a tobacco etch virus, aspartic, glutamic, metallo, cysteine, serine, or threonine protease.
18. The system of any one of embodiments 1 to 17, wherein the plasma membrane polypeptide comprises rhodopsin.
19. The system of any one of embodiments 1 to 18, wherein expression of the plasma membrane construct is inducible.
20. The system of any one of embodiments 1 to 19, wherein expression of the plasma membrane construct is induced in response to doxycycline.
21. The system of any one of embodiments 1 to 20, wherein the plasma membrane construct localizes at the plasma membrane of the eukaryotic cell after expression of the plasma membrane construct.
22. The system of any one of embodiments 1 to 21, wherein the plasma membrane polypeptide comprises an amino acid sequence at least about 90%, 95%, 97%, 98%, 99%, or 100% identical to any one of SEQ IDs 1-6.
23. The system of any one of embodiments 1 to 22, wherein expression of the reporter construct indicates an ability of a test agent to alleviate a condition.
24. The system of embodiment 23, wherein the condition is a degenerative disease.
25. The system of any one of embodiments 1 to 24, wherein the eukaryotic cell is a mammalian cell.
26. The system of embodiment 25, wherein the mammalian cell is a human cell.
27. A population of eukaryotic cells comprising the system of any one of embodiments 1 to 26.
28. A method of screening a test agent, the method comprising contacting the population of eukaryotic cells of embodiment 27 to a test agent.
29. The method of embodiment 28, wherein the test agent comprises a small molecule compound.
30. A system comprising a eukaryotic cell, wherein the eukaryotic cell comprises: (a) a plasma membrane construct comprising a plasma membrane polypeptide coupled to a transcription factor by a linker, wherein the linker is cleavable; and (b) a reporter construct comprising a promoter and a reporter gene comprising a unique molecular identifier; wherein the promoter is bound by the transcription factor upon cleavage of the linker.
31. The system of embodiment 30, further comprising a plasma membrane anchored protease capable of cleaving the linker.
32. The system of embodiment 30 or 31, wherein the plasma membrane construct is encoded by an exogenous nucleic acid.
33. The system of any one of embodiments 30 to 32, wherein the reporter construct is encoded by an exogenous nucleic acid.
34. The system of any one of embodiments 30 to 33, wherein the reporter gene further encodes a fluorescent protein or a luciferase protein.
35. The system of any one of embodiments 31 to 34, wherein the plasma membrane anchored protease is integral to the plasma membrane of the eukaryotic cell.
36. The system of any one of embodiments 31 to 34, wherein the plasma membrane anchored protease comprises a membrane tethered protease.
37. The system of embodiment 36, wherein the membrane tethered protease comprises a Pleckstrin Homology domain, platelet-derived growth factor receptor, or Lyn anchor domain.
38. The system of any one of embodiments 30 to 37, wherein the transcription factor comprises a DNA binding domain and a transcriptional activation domain.
39. The system of embodiment 38, wherein the DNA binding domain comprises a Gal4, PPR1, Lac9, zinc finger, or LexA DNA binding domain.
40. The system of embodiment 38 or 39, wherein the transcriptional activation domain comprises a VP64, VPr, p65, Rta, or VP16 activation domain.
41. The system of any one of embodiments 30 to 40, wherein the linker comprises a flexible amino acid linker.
42. The system of any one of embodiments 30 to 41, wherein the linker has a length of about 2 to about 31 amino acids.
43. The system of any one of embodiments 30 to 42, wherein the plasma membrane anchored protease comprises a tobacco etch virus, aspartic, glutamic, metallo, cysteine, serine, or threonine protease.
44. The system of any one of embodiments 30 to 43, wherein the plasma membrane polypeptide comprises rhodopsin.
45. The system of any one of embodiments 30 to 44, wherein expression of the plasma membrane construct is inducible.
46. The system of any one of embodiments 30 to 45, wherein expression of the plasma membrane construct is induced by doxycycline.
47. The system of embodiment 45 or 46, wherein the plasma membrane polypeptide localizes at the plasma membrane of the eukaryotic cell after expression of the plasma membrane polypeptide.
48. The system of any one of embodiments 30 to 47, wherein the plasma membrane polypeptide comprises an amino acid sequence at least about 90%, 95%, 97%, 98%, 99%, or 100% identical to any one of SEQ IDs 1-6.
49. The system of any one of embodiments 30 to 48, wherein expression of the reporter construct indicates an ability of a test agent to alleviate a condition.
50. The system of embodiment 49, wherein the condition is a degenerative disease.
51. The system of any one of embodiments 30 to 50, wherein the eukaryotic cell is a mammalian cell.
52. The system of embodiment 51, wherein the mammalian cell is a human cell.
53. A population of eukaryotic cells comprising the system of any one of embodiments 30 to 52.
54. A method of screening a test agent, the method comprising contacting the population of eukaryotic cells of embodiment 53 to a test agent.
55. The method of embodiment 54, wherein the test agent comprises a small molecule compound.

TABLE 1
Sequences of plasma membrane peptides

| SEQ | | | |
|---|---|---|---|
| ID | | | |
| NO | Polypeptide | Source | Sequence |
| 1 | Rhodopsin | Human | MNGTEGPNFYVPFSNATGVVRSPFEYPQYYLAEPWQ |
| | | | FSMLAAYMFLLIVLGFPINFLTLYVTVQHKKLRTPLN |
| | | | YILLNLAVADLFMVLGGFTSTLYTSLHGYFVFGPTGC |
| | | | NLEGFFATLGGEIALWSLVVLAIERYVVVCKPMSNFR |
| | | | FGENHAIMGVAFTWVMALACAAPPLAGWSRYIPEG |
| | | | LQCSCGIDYYTLKPEVNNESFVIYMFVVHFTIPMIIIFF |
| | | | CYGQLVFTVKEAAAQQQESATTQKAEKEVTRMVIIM |
| | | | VIAFLICWVPYASVAFYIFTHQGSNFGPIFMTIPAFFA |
| | | | KSAAIYNPVIYIMMNKQFRNCMLTTICCGKNPLGDD |
| | | | EASATVSKTETSQVAPA |
| 2 | Rhodopsin | Bonobo | MNGTEGPNFYVPFSNATGVVRSPFEYPQYYLAEPWQ |
| | | | FSMLAAYMFLLIVLGFPINFLTLYVTVQHKKLRTPLN |
| | | | YILLNLAVADLFMVLGGFTSTLYTSLHGYFVFGPTGC |
| | | | NLEGFFATLGGEIALWSLVVLAIERYVVVCKPMSNFR |
| | | | FGENHAIMGVAFTWVMALACAAPPLAGWSRYIPEG |
| | | | LQCSCGIDYYTLKPEVNNESFVIYMFVVHFTIPMIIIFF |
| | | | CYGQLVFTVKEAAAQQQESATTQKAEKEVTRMVIIM |
| | | | VIAFLICWVPYASVAFYIFTHQGSNFGPIFMTIPAFFA |
| | | | KSAAIYNPVIYIMMNKQFRNCMLTTICCGKNPLGDD |
| | | | EASATVSKTETSQVAPA |
| 3 | Rhodopsin | Rat | MNGTEGPNFYVPFSNITGVVRSPFEQPQYYLAEPWQF |
| | | | SMLAAYMFLLIVLGFPINFLTLYVTVQHKKLRTPLNY |
| | | | ILLNLAVADLFMVFGGFTTTLYTSLHGYFVFGPTGCN |
| | | | LEGFFATLGGEIGLWSLVVLAIERYVVVCKPMSNFRF |
| | | | GENHAIMGVAFTWVMALACAAPPLVGWSRYIPEGM |
| | | | QCSCGIDYYTLKPEVNNESFVIYMFVVHFTIPMIVIFF |
| | | | CYGQLVFTVKEAAAQQQESATTQKAEKEVTRMVIIM |
| | | | VIFFLICWLPYASVAMYIFTHQGSNFGPIFMTLPAFFA |
| | | | KTASIYNPIIYIMMNKQFRNCMLTSLCCGKNPLGDDE |
| | | | ASATASKTETSQVAPA |
| 4 | Rhodopsin | Mouse | MNGTEGPNFYVPFSNVTGVVRSPFEQPQYYLAEPWQ |
| | | | FSMLAAYMFLLIVLGFPINFLTLYVTVQHKKLRTPLN |
| | | | YILLNLAVADLFMVFGGFTTTLYTSLHGYFVFGPTGC |
| | | | NLEGFFATLGGEIALWSLVVLAIERYVVVCKPMSNFR |
| | | | FGENHAIMGVVFTWIMALACAAPPLVGWSRYIPEGM |
| | | | QCSCGIDYYTLKPEVNNESFVIYMFVVHFTIPMIVIFF |
| | | | CYGQLVFTVKEAAAQQQESATTQKAEKEVTRMVIIM |
| | | | VIFFLICWLPYASVAFYIFTHQGSNFGPIFMTLPAFFA |
| | | | KSSSIYNPVIYIMLNKQFRNCMLTTLCCGKNPLGDDD |
| | | | ASATASKTETSQVAPA |
| 5 | Rhodopsin | Horse | MINNAPNLPGADSARSLGRGGHFIRPWGGSEPGVVQ |
| | | | PEPWVAELRPQQYSWVGDAHGAATRAAAMNGTEG |
| | | | PNFYVPFSNKTGVVRSPFEYPQYYLAEPWQFSMLAA |
| | | | YMFLLIVLGFPINFLTLYVTVQHKKLRTPLNYILLNLA |
| | | | VADLFMVFGGFTTTLYTSLHGYFVFGPTGCNVEGFF |
| | | | ATLGGEIALWSLVVLAIERYVVVCKPMSNFRFGENH |
| | | | AIMGVAFTWVMALACAAPPLVGWSRYIPEGMQCSC |
| | | | GIDYYTLKPEVNNESFVIYMFVVHFTIPLIVIFFCYGQ |
| | | | LVFTVKEAAAQQQESATTQKAEKEVTRMVIIMVIAF |
| | | | LICWVPYASVAFYIFTHQGSNFGPIFMTLPAFFAKSAA |
| | | | IYNPVIYIMMNKQFRNCMITTLCCGKNPLGDDEASTT |
| | | | ASKTETSQVAPA |
| 6 | Rhodopsin | Gorilla | MNGTEGPNFYVPFSNATGVVRSPFEYPQYYLAEPWQ |
| | | | FSMLAAYMFLLIVLGFPINFLTLYVTVQHKKLRTPLN |
| | | | YILLNLAVADLFMVLGGFTSTLYTSLHGYFVFGPTGC |
| | | | NLEGFFATLGGEIALWSLVVLAIERYVVVCKPMSNFR |
| | | | FGENHAIMGVAFTWVMALACAAPPLAGWSRYIPEG |
| | | | LQCSCGIDYYTLKPEVNNESFVIYMFVVHFTIPMIIIFF |
| | | | CYGQLVFTVKEAAAQQQESATTQKAEKEVTRMVIIM |
| | | | VIAFLICWVPYASVAFYIFTHQGSNFGPIFMTIPAFFA |
| | | | KSAAIYNPVIYIMMNKQFRNCMLTTICCGKNPLGDD |
| | | | EASATVSKTETSQVAPA |
| 7 | cystic | Human | MQRSPLEKASVVSKLFFSWTRPILRKGYRQRLELSDI |
| | fibrosis | | YQIPSVDSADNLSEKLEREWDRELASKKNPKLINALR |
| | transmembrane | | RCFFWRFMFYGIFLYLGEVTKAVQPLLLGRIIASYDP |
| | conductance | | DNKEERSIAIYLGIGLCLLFIVRTLLLHPAIFGLHHIGM |
| | regulator | | QMRIAMFSLIYKKTLKLSSRVLDKISIGQLVSLLSNNL |
| | (CFTR) | | NKFDEGLALAHFVWIAPLQVALLMGLIWELLQASAF |
| | | | CGLGFLIVLALFQAGLGRMMMKYRDQRAGKISERLV |
| | | | ITSEMIENIQSVKAYCWEEAMEKMIENLRQTELKLTR |
| | | | KAAYVRYFNSSAFFFSGFFVVFLSVLPYALIKGIILRKI |
| | | | FTTISFCIVLRMAVTRQFPWAVQTWYDSLGAINKIQD |
| | | | FLQKQEYKTLEYNLTTTEVVMENVTAFWEEGFGELF |
| | | | EKAKQNNNNRKTSNGDDSLFFSNFSLLGTPVLKDINF |
| | | | KIERGQLLAVAGSTGAGKTSLLMVIMGELEPSEGKIK |
| | | | HSGRISFCSQFSWIMPGTIKENIIFGVSYDEYRYRSVIK |
| | | | ACQLEEDISKFAEKDNIVLGEGGITLSGGQRARISLAR |
| | | | AVYKDADLYLLDSPFGYLDVLTEKEIFESCVCKLMA |
| | | | NKTRILVTSKMEHLKKADKILILHEGSSYFYGTFSELQ |
| | | | NLQPDFSSKLMGCDSFDQFSAERRNSILTETLHRFSLE |
| | | | GDAPVSWTETKKQSFKQTGEFGEKRKNSILNPINSIR |
| | | | KFSIVQKTPLQMNGIEEDSDEPLERRLSLVPDSEQGEA |
| | | | ILPRISVISTGPTLQARRRQSVLNLMTHSVNQGQNIHR |
| | | | KTTASTRKVSLAPQANLTELDIYSRRLSQETGLEISEEI |
| | | | NEEDLKECFFDDMESIPAVTTWNTYLRYITVHKSLIF |
| | | | VLIWCLVIFLAEVAASLVVLWLLGNTPLQDKGNSTH |
| | | | SRNNSYAVIITSTSSYYVFYIYVGVADTLLAMGFFRG |
| | | | LPLVHTLITVSKILHHKMLHSVLQAPMSTLNTLKAGG |
| | | | ILNRFSKDIAILDDLLPLTIFDFIQLLLIVIGAIAVVAVL |
| | | | QPYIFVATVPVIVAFIMLRAYFLQTSQQLKQLESEGRS |
| | | | PIFTHLVTSLKGLWTLRAFGRQPYFETLFHKALNLHT |
| | | | ANWFLYLSTLRWFQMRIEMIFVIFFIAVTFISILTTGEG |
| | | | EGRVGIILTLAMNIMSTLQWAVNSSIDVDSLMRSVSR |
| | | | VFKFIDMPTEGKPTKSTKPYKNGQLSKVMIIENSHVK |
| | | | KDDIWPSGGQMTVKDLTAKYTEGGNAILENISFSISP |
| | | | GQRVGLLGRTGSGKSTLLSAFLRLLNTEGEIQIDGVS |
| | | | WDSITLQQWRKAFGVIPQKVFIFSGTFRKNLDPYEQ |
| | | | WSDQEIWKVADEVGLRSVIEQFPGKLDFVLVDGGCV |
| | | | LSHGHKQLMCLARSVLSKAKILLLDEPSAHLDPVTY |
| | | | QIIRRTLKQAFADCTVILCEHRIEAMLECQQFLVIEEN |
| | | | KVRQYDSIQKLLNERSLFRQAISPSDRVKLFPHRNSSK |
| | | | CKSKPQIAALKEETEEEVQDTRL |
| 8 | Glucagon- | Human | MAGAPGPLRLALLLLGMVGRAGPRPQGATVSLWET |
| | like peptide | | VQKWREYRRQCQRSLTEDPPPATDLFCNRTFDEYAC |
| | 1 receptor | | WPDGEPGSFVNVSCPWYLPWASSVPQGHVYRFCTA |
| | (GLP-1R) | | EGLWLQKDNSSLPWRDLSECEESKRGERSSPEEQLLF |
| | | | LYIIYTVGYALSFSALVIASAILLGFRHLHCTRNYIHL |
| | | | NLFASFILRALSVFIKDAALKWMYSTAAQQHQWDGL |
| | | | LSYQDSLSCRLVFLLMQYCVAANYYWLLVEGVYLY |
| | | | TLLAFSVLSEQWIFRLYVSIGWGVPLLFVVPWGIVKY |
| | | | LYEDEGCWTRNSNMNYWLIIRLPILFAIGVNFLIFVRV |
| | | | ICIVVSKLKANLMCKTDIKCRLAKSTLTLIPLLGTHEV |
| | | | IFAFVMDEHARGTLRFIKLFTELSFTSFQGLMVAILYC |
| | | | FVNNEVQLEFRKSWERWRLEHLHIQRDSSMKPLKCP |
| | | | TSSLSSGATAGSSMYTATCQASCS |
| | | | MNSTGHLQDAPNATSLHVPHSQEGNSTSLQEGLQDL |
| 9 | GPR75 | Human | IHTATLVTCTFLLAVIFCLGSYGNFIVFLSFFDPAFRKF |
| | | | RTNFDFMILNLSFCDLFICGVTAPMFTFVLFFSSASSIP |
| | | | DAFCFTFHLTSSGFIIMSLKTVAVIALHRLRMVLGKQ |
| | | | PNRTASFPCTVLLTLLLWATSFTLATLATLKTSKSHL |
| | | | CLPMSSLIAGKGKAILSLYVVDFTFCVAVVSVSYIMI |
| | | | AQTLRKNAQVRKCPPVITVDASRPQPFMGVPVQGGG |
| | | | DPIQCAMPALYRNQNYNKLQHVQTRGYTKSPNQLV |
| | | | TPAASRLQLVSAINLSTAKDSKAVVTCVIIVLSVLVCC |
| | | | LPLGISLVQVVLSSNGSFILYQFELFGFTLIFFKSGLNP |
| | | | FIYSRNSAGLRRKVLWCLQYIGLGFFCCKQKTRLRA |
| | | | MGKGNLEVNRNKSSHHETNSAYMLSPKPQKKFVDQ |
| | | | ACGPSHSKESMVSPKISAGHQHCGQSSSTPINTRIEPY |
| | | | YSIYNSSPSQEESSPCNLQPVNSFGFANSYIAMHYHTT |
| | | | NDLVQEYDSTSAKQIPVPSV |
| 10 | GIPR | Human | MTTSPILQLLLRLSLCGLLLQRAETGSKGQTAGELYQ |
| | | | RWERYRRECQETLAAAEPPSGLACNGSFDMYVCWD |
| | | | YAAPNATARASCPWYLPWHHHVAAGFVLRQCGSDG |
| | | | QWGLWRDHTQCENPEKNEAFLDQRLILERLQVMYT |
| | | | VGYSLSLATLLLALLILSLFRRLHCTRNYIHINLFTSF |
| | | | MLRAAAILSRDRLLPRPGPYLGDQALALWNQALAAC |
| | | | RTAQIVTQYCVGANYTWLLVEGVYLHSLLVLVGGSE |
| | | | EGHFRYYLLLGWGAPALFVIPWVIVRYLYENTQCWE |
| | | | RNEVKAIWWIIRTPILMTILINFLIFIRILGILLSKLRTRQ |
| | | | MRCRDYRLRLARSTLTLVPLLGVHEVVFAPVTEEQA |
| | | | RGALRFAKLGFEIFLSSFQGFLVSVLYCFINKEVQSEI |
| | | | RRGWHHCRLRRSLGEEQRQLPERAFRALPSGSGPGE |
| | | | VPTSRGLSSGTLPGPGNEASRELESYC |
| 11 | MC1R | Human | MAVQGSQRRLLGSLNSTPTAIPQLGLAANQTGARCL |
| | | | EVSISDGLFLSLGLVSLVENALVVATIAKNRNLHSPM |
| | | | YCFICCLALSDLLVSGSNVLETAVILLLEAGALVARA |
| | | | AVLQQLDNVIDVITCSSMLSSLCFLGAIAVDRYISIFY |
| | | | ALRYHSIVTLPRARRAVAAIWVASVVFSTLFIAYYDH |
| | | | VAVLLCLVVFFLAMLVLMAVLYVHMLARACQHAQ |
| | | | GIARLHKRQRPVHQGFGLKGAVTLTILLGIFFLCWGP |
| | | | FFLHLTLIVLCPEHPTCGCIFKNFNLFLALIICNAIIDPLI |
| | | | YAFHSQELRRTLKEVLTCSW |
| 12 | MC2R | Human | MKHIINSYENINNTARNNSDCPRVVLPEEIFFTISIVGV |
| | | | LENLIVLLAVFKNKNLQAPMYFFICSLAISDMLGSLY |
| | | | KILENILIILRNMGYLKPRGSFETTADDIIDSLFVLSLL |
| | | | GSIFSLSVIAADRYITIFHALRYHSIVTMRRTVVVLTVI |
| | | | WTFCTGTGITMVIFSHHVPTVITFTSLFPLMLVFILCL |
| | | | YVHMFLLARSHTRKISTLPRANMKGAITLTILLGVFIF |
| | | | CWAPFVLHVLLMTFCPSNPYCACYMSLFQVNGMLI |
| | | | MCNAVIDPFIYAFRSPELRDAFKKMIFCSRYW |
| 13 | MC3R | Human | MNASCCLPSVQPTLPNGSEHLQAPFFSNQSSSAFCEQ |
| | | | VFIKPEVFLSLGIVSLLENILVILAVVRNGNLHSPMYF |
| | | | FLCSLAVADMLVSVSNALETIMIAIVHSDYLTFEDQFI |
| | | | QHMDNIFDSMICISLVASICNLLAIAVDRYVTIFYALR |
| | | | YHSIMTVRKALTLIVAIWVCCGVCGVVFIVYSESKM |
| | | | VIVCLITMFFAMMLLMGTLYVHMFLFARLHVKRIAA |
| | | | LPPADGVAPQQHSCMKGAVTITILLGVFIFCWAPFFL |
| | | | HLVLIITCPTNPYCICYTAHFNTYLVLIMCNSVIDPLIY |
| | | | AFRSLELRNTFREILCGCNGMNLG |
| 14 | MC4R | Human | MVNSTHRGMHTSLHLWNRSSYRLHSNASESLGKGY |
| | | | SDGGCYEQLFVSPEVFVTLGVISLLENILVIVAIAKNK |
| | | | NLHSPMYFFICSLAVADMLVSVSNGSETIVITLLNSTD |
| | | | TDAQSFTVNIDNVIDSVICSSLLASICSLLSIAVDRYFTI |
| | | | FYALQYHNIMTVKRVGIIISCIWAACTVSGILFIIYSDS |
| | | | SAVIICLITMFFTMLALMASLYVHMFLMARLHIKRIA |
| | | | VLPGTGAIRQGANMKGAITLTILIGVFVVCWAPFFLH |
| | | | LIFYISCPQNPYCVCFMSHFNLYLILIMCNSIIDPLIYAL |
| | | | RSQELRKTFKEIICCYPLGGLCDLSSRY |
| 15 | MC5R | Human | MNSSFHLHFLDLNLNATEGNLSGPNVKNKSSPCEDM |
| | | | GIAVEVFLTLGVISLLENILVIGAIVKNKNLHSPMYFF |
| | | | VCSLAVADMLVSMSSAWETITIYLLNNKHLVIADAF |
| | | | VRHIDNVFDSMICISVVASMCSLLAIAVDRYVTIFYAL |
| | | | RYHHIMTARRSGAIIAGIWAFCTGCGIVFILYSESTYVI |
| | | | LCLISMFFAMLFLLVSLYIHMFLLARTHVKRIAALPG |
| | | | ASSARQRTSMQGAVTVTMLLGVFTVCWAPFFLHLTL |
| | | | MLSCPQNLYCSRFMSHFNMYLILIMCNSVMDPLIYAF |
| | | | RSQEMRKTFKEIICCRGFRIACSFPRRD |
| 16 | GCGR | Human | MPPCQPQRPLLLLLLLLACQPQVPSAQVMDFLFEKW |
| | | | KLYGDQCHHNLSLLPPPTELVCNRTFDKYSCWPDTP |
| | | | ANTTANISCPWYLPWHHKVQHRFVFKRCGPDGQWV |
| | | | RGPRGQPWRDASQCQMDGEEIEVQKEVAKMYSSFQ |
| | | | VMYTVGYSLSLGALLLALAILGGLSKLHCTRNAIHA |
| | | | NLFASFVLKASSVLVIDGLLRTRYSQKIGDDLSVSTW |
| | | | LSDGAVAGCRVAAVFMQYGIVANYCWLLVEGLYLH |
| | | | NLLGLATLPERSFFSLYLGIGWGAPMLFVVPWAVVK |
| | | | CLFENVQCWTSNDNMGFWWILRFPVFLAILINFFIFV |
| | | | RIVQLLVAKLRARQMHHTDYKFRLAKSTLTLIPLLGV |
| | | | HEVVFAFVTDEHAQGTLRSAKLFFDLFLSSFQGLLVA |
| | | | VLYCFLNKEVQSELRRRWHRWRLGKVLWEERNTSN |
| | | | HRASSSPGHGPPSKELQFGRGGGSQDSSAETPLAGGL |
| | | | PRLAESPF |
| 17 | DRD2 | Human | MDPLNLSWYDDDLERQNWSRPFNGSDGKADRPHYN |
| | | | YYATLLTLLIAVIVFGNVLVCMAVSREKALQTTTNY |
| | | | LIVSLAVADLLVATLVMPWVVYLEVVGEWKFSRIHC |
| | | | DIFVTLDVMMCTASILNLCAISIDRYTAVAMPMLYNT |
| | | | RYSSKRRVTVMISIVWVLSFTISCPLLFGLNNADQNE |
| | | | CIIANPAFVVYSSIVSFYVPFIVTLLVYIKIYIVLRRRRK |
| | | | RVNTKRSSRAFRAHLRAPLKGNCTHPEDMKLCTVIM |
| | | | KSNGSFPVNRRRVEAARRAQELEMEMLSSTSPPERTR |
| | | | YSPIPPSHHQLTLPDPSHHGLHSTPDSPAKPEKNGHA |
| | | | KDHPKIAKIFEIQTMPNGKTRTSLKTMSRRKLSQQKE |
| | | | KKATQMLAIVLGVFIICWLPFFITHILNIHCDCNIPPVL |
| | | | YSAFTWLGYVNSAVNPIIYTTFNIEFRKAFLKILHC |
| 18 | HTR2A | Human | MDILCEENTSLSSTTNSLMQLNDDTRLYSNDFNSGEA |
| | | | NTSDAFNWTVDSENRTNLSCEGCLSPSCLSLLHLQEK |
| | | | NWSALLTAVVIILTIAGNILVIMAVSLEKKLQNATNY |
| | | | FLMSLAIADMLLGFLVMPVSMLTILYGYRWPLPSKL |
| | | | CAVWIYLDVLFSTASIMHLCAISLDRYVAIQNPIHHSR |
| | | | FNSRTKAFLKIIAVWTISVGISMPIPVFGLQDDSKVFK |
| | | | EGSCLLADDNFVLIGSFVSFFIPLTIMVITYFLTIKSLQ |
| | | | KEATLCVSDLGTRAKLASFSFLPQSSLSSEKLFQRSIH |
| | | | REPGSYTGRRTMQSISNEQKACKVLGIVFFLFVVMW |
| | | | CPFFITNIMAVICKESCNEDVIGALLNVFVWIGYLSSA |
| | | | VNPLVYTLFNKTYRSAFSRYIQCQYKENKKPLQLILV |
| | | | NTIPALAYKSSQLQMGQKKNSKQDAKTTDNDCSMV |
| | | | ALGKQHSEEASKDNSDGVNEKVSCV |
| 19 | ABCA4 | Human | MGFVRQIQLLLWKNWTLRKRQKIRFVVELVWPLSLF |
| | | | LVLIWLRNANPLYSHHECHFPNKAMPSAGMLPWLQ |
| | | | GIFCNVNNPCFQSPTPGESPGIVSNYNNSILARVYRDF |
| | | | QELLMNAPESQHLGRIWTELHILSQFMDTLRTHPERI |
| | | | AGRGIRIRDILKDEETLTLFLIKNIGLSDSVVYLLINSQ |
| | | | VRPEQFAHGVPDLALKDIACSEALLERFIIFSQRRGAK |
| | | | TVRYALCSLSQGTLQWIEDTLYANVDFFKLFRVLPTL |
| | | | LDSRSQGINLRSWGGILSDMSPRIQEFIHRPSMQDLL |
| | | | WVTRPLMQNGGPETFTKLMGILSDLLCGYPEGGGSR |
| | | | VLSFNWYEDNNYKAFLGIDSTRKDPIYSYDRRTTSFC |
| | | | NALIQSLESNPLTKIAWRAAKPLLMGKILYTPDSPAA |
| | | | RRILKNANSTFEELEHVRKLVKAWEEVGPQIWYFFD |
| | | | NSTQMNMIRDTLGNPTVKDFLNRQLGEEGITAEAILN |
| | | | FLYKGPRESQADDMANFDWRDIFNITDRTLRLVNQY |
| | | | LECLVLDKFESYNDETQLTQRALSLLEENMFWAGVV |
| | | | FPDMYPWTSSLPPHVKYKIRMDIDVVEKTNKIKDRY |
| | | | WDSGPRADPVEDFRYIWGGFAYLQDMVEQGITRSQ |
| | | | VQAEAPVGIYLQQMPYPCFVDDSFMIILNRCFPIFMV |
| | | | LAWIYSVSMTVKSIVLEKELRLKETLKNQGVSNAVI |
| | | | WCTWFLDSFSIMSMSIFLLTIFIMHGRILHYSDPFILFL |
| | | | FLLAFSTATIMLCFLLSTFFSKASLAAACSGVIYFTLY |
| | | | LPHILCFAWQDRMTAELKKAVSLLSPVAFGFGTEYL |
| | | | VRFEEQGLGLQWSNIGNSPTEGDEFSFLLSMQMMLL |
| | | | DAAVYGLLAWYLDQVFPGDYGTPLPWYFLLQESYW |
| | | | LGGEGCSTREERALEKTEPLTEETEDPEHPEGIHDSFF |
| | | | EREHPGWVPGVCVKNLVKIFEPCGRPAVDRLNITFYE |
| | | | NQITAFLGHNGAGKTTTLSILTGLLPPTSGTVLVGGR |
| | | | DIETSLDAVRQSLGMCPQHNILFHHLTVAEHMLFYA |
| | | | QLKGKSQEEAQLEMEAMLEDTGLHHKRNEEAQDLS |
| | | | GGMQRKLSVAIAFVGDAKVVILDEPTSGVDPYSRRSI |
| | | | WDLLLKYRSGRTIIMSTHHMDEADLLGDRIAIIAQGR |
| | | | LYCSGTPLFLKNCFGTGLYLTLVRKMKNIQSQRKGSE |
| | | | GTCSCSSKGFSTTCPAHVDDLTPEQVLDGDVNELMD |
| | | | VVLHHVPEAKLVECIGQELIFLLPNKNFKHRAYASLF |
| | | | RELEETLADLGLSSFGISDTPLEEIFLKVTEDSDSGPLF |
| | | | AGGAQQKRENVNPRHPCLGPREKAGQTPQDSNVCSP |
| | | | GAPAAHPEGQPPPEPECPGPQLNTGTQLVLQHVQAL |
| | | | LVKRFQHTIRSHKDFLAQIVLPATFVFLALMLSIVIPPF |
| | | | GEYPALTLHPWIYGQQYTFFSMDEPGSEQFTVLADV |
| | | | LLNKPGFGNRCLKEGWLPEYPCGNSTPWKTPSVSPNI |
| | | | TQLFQKQKWTQVNPSPSCRCSTREKLTMLPECPEGA |
| | | | GGLPPPQRTQRSTEILQDLTDRNISDFLVKTYPALIRSS |
| | | | LKSKFWVNEQRYGGISIGGKLPVVPITGEALVGFLSD |
| | | | LGRIMNVSGGPITREASKEIPDFLKHLETEDNIKVWFN |
| | | | NKGWHALVSFLNVAHNAILRASLPKDRSPEEYGITVI |
| | | | SQPLNLTKEQLSEITVLTTSVDAVVAICVIFSMSFVPA |
| | | | SFVLYLIQERVNKSKHLQFISGVSPTTYWVTNFLWDI |
| | | | MNYSVSAGLVVGIFIGFQKKAYTSPENLPALVALLLL |
| | | | YGWAVIPMMYPASFLFDVPSTAYVALSCANLFIGINS |
| | | | SAITFILELFENNRTLLRFNAVLRKLLIVFPHFCLGRGL |
| | | | IDLALSQAVTDVYARFGEEHSANPFHWDLIGKNLFA |
| | | | MVVEGVVYFLLTLLVQRHFFLSQWIAEPTKEPIVDED |
| | | | DDVAEERQRIITGGNKTDILRLHELTKIYPGTSSPAVD |
| | | | RLCVGVRPGECFGLLGVNGAGKTTTFKMLTGDTTVT |
| | | | SGDATVAGKSILTNISEVHQNMGYCPQFDAIDELLTG |
| | | | REHLYLYARLRGVPAEEIEKVANWSIKSLGLTVYAD |
| | | | CLAGTYSGGNKRKLSTAIALIGCPPLVLLDEPTTGMD |
| | | | PQARRMLWNVIVSIIREGRAVVLTSHSMEECEALCTR |
| | | | LAIMVKGAFRCMGTIQHLKSKFGDGYIVTMKIKSPK |
| | | | DDLLPDLNPVEQFFQGNFPGSVQRERHYNMLQFQVS |
| | | | SSSLARIFQLLLSHKDSLLIEEYSVTQTTLDQVFVNFA |
| | | | KQQTESHDLPLHPRAAGASRQAQD |

TABLE 2
Sequences of plasma membrane construct linkers
and domains thereof

| SEQ | | | |
|---|---|---|---|
| ID | Linker/ | | |
| NO | Domain | Source | Sequence |
| 20 | Full length, | Synthetic | GSENLYFQSGS |
| | TCS(S) | | |
| 21 | Full length, | Synthetic | GSENLYFQYGS |
| | TCS(Y) | | |

Protease cleavage site

| | | | |
|---|---|---|---|
| 22 | Protease | Synthetic | ENLYFQ(X) |
| | cleavage | | |
| | site | | |
| 23 | Protease | Synthetic | ENLYFQS |
| | cleavage | | |
| | site, TCS(S) | | |
| 24 | Protease | Synthetic | ENLYFQY |
| | cleavage | | |
| | site, | | |
| | TCS(Y) | | |

TABLE 3
Sequences of transcription factors and domains thereof

| | | | |
|---|---|---|---|
| SEQ | | | |
| ID | | | |
| NO | Domain | Source | Sequence |

DNA binding domain (DBD)

| | | | |
|---|---|---|---|
| 25 | zinc finger | synthetic | ARPGERPFQCRICMANFSRNFILQRHTRTHTGEKPFQ |
| | (RARR) | | CRICMRNFSDRANLRRHLRTHTGEKPFQCRICMRNF |
| | | | SRHDQLTRHLKTHLRGS |
| 26 | gal4 | yeast | MKLLSSIEQACDICRLKKLKCSPEKPKCAKCLKNNW |
| | | | ECRYSPKTKRSPLTRAHLTEVESRLERLEQLFLLIFPR |
| | | | EDLDMILKMDSLQDIKALLTGLFVQDNVNKDAVTD |
| | | | RLASVETDMPLTLRQHRISATSSSEESSNKGQRQLTV |
| | | | S |
| 27 | PPR1 | yeast | MKSRTACKRCRLKKIKCDQEFPSCKRCAKLEVPCVS |
| | | | LDPATGKDVPRSYVFFLEDRLAVMMRVLKEYGVDP |
| | | | TKIRGNIPATSDDEPFDLKKYSSVSSLGEEGILPHNGL |
| | | | LADYLVQKGNSMASSAITSKSMASPQTINVQRKEFL |
| | | | VNSKKQDGSALLPETGS |
| 28 | Lac9 | yeast | MNKKSSEVMHQACKRCRLKKIKCDQEFPSCKRCLK |
| | | | YNLDCVYSPQVVRTPLTRAHLTEMENRVAELEQFL |
| | | | KELFPVWDIDRLLQQKDTYRIRELLTMGSTNTVPGL |
| | | | ASNNIDSSLEQPVAFGTAQPAQSLSTDPAVQSQAYP |
| | | | MQPV |
| 29 | LexA | e. coli | MKALTARQQEVEDLIRDHISQTGMPPTRAEIAQRLG |
| | | | FRSPNAAEEHLKALARKGVIEIVSGASRGIRLLQEEE |
| | | | EGLPLVGRVAAGEPLLAQQHIEGHYQVDPSLFKPNA |
| | | | DFLLRVSGMSMKDIGIMDGDLLAVHKTQDVRNGQ |
| | | | VVVARIDDEVTVKRLKKQGNKVELLPENSEFKPIVV |
| | | | DLRQQSFTIEGLAVGVIRNGDWL |

Transactivator

| | | | |
|---|---|---|---|
| 30 | VP64 | synthetic | DALDDFDLDMLGSDALDDFDLDMLGSDALDDFDL |
| | | | DMLGSDALDDFDLDML |
| 31 | VPr | synthetic | GKPIPNPLLGLDSTDALDDFDLDMLGSDALDDFDLD |
| | | | MLGSDALDDFDLDMLGSDALDDFDLDMLGSPKKK |
| | | | RKVGSQYLPDTDDRHRIEEKRKRTYETFKSIMKKSP |
| | | | FSGPTDPRPPPRRIAVPSRSSASVPKPAPQPYPFTSSLS |
| | | | TINYDEFPTMVFPSGQISQASALAPAPPQVLPQAPAP |
| | | | APAPAMVSALAQAPAPVPVLAPGPPQAVAPPAPKPT |
| | | | QAGEGTLSEALLQLQFDDEDLGALLGNSTDPAVFTD |
| | | | LASVDNSEFQQLLNQGIPVAPHTTEPMLMEYPEAIT |
| | | | RLVTGAQRPPDPAPAPLGAPGLPNGLLSGDEDFSSIA |
| | | | DMDFSALLSQISSGSGSGSRDSREGMFLPKPEAGSAI |
| | | | SDVFEGREVCQPKRIRPFHPPGSPWANRPLPASLAPT |
| | | | PTGPVHEPVGSLTPAPVPQPLDPAPAVTPEASHLLED |
| | | | PDEETSQAVKALREMADTVIPQKEEAAICGQMDLSH |
| | | | PPPRGHLDELTTTLESMTEDLNLDSPLTPELNEILDTF |
| | | | LNDECLLHAMHISTGLSIFDTSLFNSACKNWFSSLSH |
| | | | FVIHL |
| 32 | p65 | human | QYLPDTDDRHRIEEKRKRTYETFKSIMKKSPFSGPTD |
| | | | PRPPPRRIAVPSRSSASVPKPAPQPYPFTSSLSTINYDE |
| | | | FPTMVFPSGQISQASALAPAPPQVLPQAPAPAPAPA |
| | | | MVSALAQAPAPVPVLAPGPPQAVAPPAPKPTQAGE |
| | | | GTLSEALLQLQFDDEDLGALLGNSTDPAVFTDLASV |
| | | | DNSEFQQLLNQGIPVAPHTTEPMLMEYPEAITRLVT |
| | | | GAQRPPDPAPAPLGAPGLPNGLLSGDEDFSSIADMD |
| | | | FSALLSQISS |
| 33 | Rta | EPV | RDSREGMFLPKPEAGSAISDVFEGREVCQPKRIRPFH |
| | | | PPGSPWANRPLPASLAPTPTGPVHEPVGSLTPAPVPQ |
| | | | PLDPAPAVTPEASHLLEDPDEETSQAVKALREMADT |
| | | | VIPQKEEAAICGQMDLSHPPPRGHLDELTTTLESMTE |
| | | | DLNLDSPLTPELNEILDTFLNDECLLHAMHISTGLSIF |
| | | | DTSLF |
| 34 | VP16 | HSV | DALDDFDLDML |

TABLE 4
Sequences of plasma membrane anchored proteases and domains thereof

| | | | |
|---|---|---|---|
| SEQ | | | |
| ID | | | |
| NO | Domain | Source | Sequence |

Anchor

| | | | |
|---|---|---|---|
| 35 | Lyn11 | human | MGCIKSKGKDSAGA |
| 36 | PHDeltal | human | MHGLQDDEDLQALLKGSQLLKVKSSSWRRERFYKL |
| | Pleckstrin | | QEDCKTIWQESRKVMRTPESQLFSIEDIQEVRMGHR |
| | Homology | | TEGLEKFARDVPEDRCFSIVFKDQRNTLDLIAPSPAD |
| | domain-1 | | AQHWVLGLHKIIHHSGS |
| 37 | PHDelta3 | human | MRAMLRGSRLRKIRSRTWHKERLYRLQEDGLSVWF |
| | Pleckstrin | | QRRIPRAPSQHIFFVQHIEAVREGHQSEGLRRFGGAF |
| | Homology | | APARCLTIAFKGRRKNLDLAAPTAEEAQRWVRGLT |
| | domain-3 | | KLRA |
| 38 | platelet- | human | AVGQDTQEVIVVPHSLPFKVVVISAILALVVLTIISLII |
| | derived | | LIMLWQKKPR |
| | growth | | |
| | factor | | |
| | receptor | | |

Protease tether

| | | | |
|---|---|---|---|
| 39 | ASPS | synthetic | ASPSNPGASNGS |
| 40 | 2xGS | synthetic | GGGGSGGGGS |

Protease

| | | | |
|---|---|---|---|
| 41 | Tobacco | TEV | SLFKGPRDYNPISSTICHLTNESDGHTTSLYGIGFGPFI |
| | Etch Virus | | ITNKHLFRRNNGTLLVQSLHGVFKVKNTTTLQQHLI |
| | (TEV) | | DGRDMIIIRMPKDFPPFPQKLKFREPQREERICLVTTN |
| | | | FQTKSMSSMVSDTSCTFPSSDGIFWKHWIQTKDGQC |
| | | | GSPLVSTRDGFIVGIHSASNFTNTNNYFTSVPKNFME |
| | | | LLTNQEAQQWVSGWRLNADSVLWGGHKVFMSKPE |
| | | | EPFQPVKEATQLMNELVYSQ |
| 42 | Tobacco | TEV | SLFKGPRDYNPISSTICHLTNESDGHTTSLYGIGFGPFI |
| | Etch Virus, | | ITNKHLFRRNNGTLLVQSLHGVFKVKNTTTLQQHLI |
| | S219V | | DGRDMIIIRMPKDFPPFPQKLKFREPQREERICLVTTN |
| | (TEV- | | FQTKSMSSMVSDTSCTFPSSDGIFWKHWIQTKDGQC |
| | S219V) | | GSPLVSTRDGFIVGIHSASNFTNTNNYFTSVPKNFME |
| | | | LLTNQEAQQWVSGWRLNADSVLWGGHKVFMVKP |
| | | | EEPFQPVKEATQLMNELVYSQ |

TABLE 5
Sequences of synthetic promoters

| SEQ | | | |
|---|---|---|---|
| ID | | | |
| NO | Promoter | Source | Sequence |
| 43 | YB_tata | synthetic | gcgattaatccatatgctctagagggtatataatgggggccactagtctactaccaga |
| | | | aagcttggtaccgagctcggatcca |
| 44 | Dox- | synthetic | tccctatcaatgatagagaggttattaagacacctcttccctatcactgatagagaact |
| | inducible | | cgacacgactggagctccctatcagtgatagagaaggtacgtcaagaacgactccc |
| | | | tatcattgatagagaaggactgtctaagacgactccctatcaatgatagagaactcga |
| | | | cagtacgctaagtccctatcactgatagagacctgaaggagtctacacatccctatca |
| | | | gtgatagagagcgcttaatcgacagaagtccctatcattgatagagaaggaacccc |
| | | | ctatataagcagagctcgtttagtgaaccgtcagatcgcctggagtcgccatccacg |
| | | | ctgttttgacctccatagatgacaccgggaccgatccagcct |

What is claimed is:

1. A system comprising a eukaryotic cell, wherein the eukaryotic cell comprises:
a. a first exogenous nucleic acid encoding a plasma membrane construct (PMC) comprising a plasma membrane polypeptide coupled to a transcription factor by a PMC linker, wherein the PMC linker comprises a protease cleavage site;
b. a second exogenous nucleic acid encoding a plasma membrane anchored protease, wherein the plasma membrane anchored protease is capable of cleaving the PMC linker; and
c. a third exogenous nucleic acid encoding a reporter construct (RC) comprising a RC promoter and a reporter gene comprising a unique molecular identifier.

2. The system of claim 1, wherein the RC promoter is bound by the transcription factor upon cleavage of the PMC linker.

3. The system of claim 1, wherein the RC promoter comprises a synthetic DNA binding domain responsive promoter.

4. The system of claim 1, wherein the RC promoter comprises a zinc finger binding site.

5. The system of claim 1, wherein expression of the plasma membrane anchored protease is driven by a constitutive promoter.

6. The system of claim 1, wherein the plasma membrane anchored protease comprises a membrane tethered protease.

7. The system of claim 6, wherein the plasma membrane anchored protease comprises a plasma membrane anchor linked to a protease by a protease tether.

8. The system of claim 7, wherein the plasma membrane anchor comprises any one of SEQ ID NOs: 35-38.

9. The system of claim 6, wherein the membrane tethered protease comprises a TEV protease or a variant TEV protease, or a functional fragment thereof.

10. The system of claim 9, wherein the membrane tethered protease comprises a sequence having at least 90% identity to SEQ ID NO: 41.

11. The system of claim 1, wherein the transcription factor comprises a DNA binding domain and a transcriptional activation domain.

12. The system of claim 11, wherein the DNA binding domain comprises a Gal4, PPR1, Lac9, zinc finger, or LexA DNA binding domain.

13. The system of claim 1, wherein the PMC linker comprises a flexible amino acid linker.

14. The system of claim 1, wherein the protease cleavage site comprises at least one of SEQ ID NOs: 22-24.

15. The system of claim 1, wherein the plasma membrane polypeptide comprises rhodopsin or a variant thereof.

16. The system of claim 1, wherein the plasma membrane polypeptide comprises cystic fibrosis transmembrane conductance regulator (CFTR) or a variant of CFTR.

17. The system of claim 1, wherein the plasma membrane polypeptide comprises a G-protein coupled receptor.

18. The system of claim 1, wherein the plasma membrane construct localizes at the plasma membrane of the eukaryotic cell after expression of the plasma membrane construct.

19. The system of claim 1, wherein a population of eukaryotic cells comprises the eukaryotic cell.

20. A method of screening a test agent, the method comprising contacting the population of eukaryotic cells of claim 19 to a test agent.


SEQUENCE LISTING (44 sequences)

SEQ ID NO: 1 - protein, 348 aa, Homo sapiens [sequence omitted]

SEQ ID NO: 2 - protein, 348 aa, Pan paniscus [sequence omitted]

SEQ ID NO: 3 - protein, 348 aa, Rattus norvegicus
  source 1..348: note = Rhodopsin
MNGTEGPNFYVPFSNITGVVRSPFEQPQYYLAEPWQFSMLAAYMFLLIVLGFPINFLTLYVTVQHKKLRTPLNYILLNLAVADLFMVFGGFTTTLYTSLHGYFVFGPTGCNLEGFFATLGGEIGLWSLVVLAIERYVVVCKPMSNFRFGENHAIMGVAFTWVMALACAAPPLVGWSRYIPEGMQCSCGIDYYTLKPEVNNESFVIYMFVVHFTIPMIVIFFCYGQLVFTVKEAAAQQQESATTQKAEKEVTRMVIIMVIFFLICWLPYASVAMYIFTHQGSNFGPIFMTLPAFFAKTASIYNPIIYIMMNKQFRNCMLTSLCCGKNPLGDDEASATASKTETSQVAPA

SEQ ID NO: 4 - protein, 348 aa, Mus musculus
  source 1..348: note = Rhodopsin
MNGTEGPNFYVPFSNVTGVVRSPFEQPQYYLAEPWQFSMLAAYMFLLIVLGFPINFLTLYVTVQHKKLRTPLNYILLNLAVADLFMVFGGFTTTLYTSLHGYFVFGPTGCNLEGFFATLGGEIALWSLVVLAIERYVVVCKPMSNFRFGENHAIMGVVFTWIMALACAAPPLVGWSRYIPEGMQCSCGIDYYTLKPEVNNESFVIYMFVVHFTIPMIVIFFCYGQLVFTVKEAAAQQQESATTQKAEKEVTRMVIIMVIFFLICWLPYASVAFYIFTHQGSNFGPIFMTLPAFFAKSSSIYNPVIYIMLNKQFRNCMLTTLCCGKNPLGDDDASATASKTETSQVAPA

SEQ ID NO: 5 - protein, 412 aa, Equus caballus
  source 1..412: note = Rhodopsin
MINNAPNLPGADSARSLGRGGHFIRPWGGSEPGVVQPEPWVAELRPQQYSWVGDAHGAATRAAAMNGTEGPNFYVPFSNKTGVVRSPFEYPQYYLAEPWQFSMLAAYMFLLIVLGFPINFLTLYVTVQHKKLRTPLNYILLNLAVADLFMVFGGFTTTLYTSLHGYFVFGPTGCNVEGFFATLGGEIALWSLVVLAIERYVVVCKPMSNFRFGENHAIMGVAFTWVMALACAAPPLVGWSRYIPEGMQCSCGIDYYTLKPEVNNESFVIYMFVVHFTIPLIVIFFCYGQLVFTVKEAAAQQQESATTQKAEKEVTRMVIIMVIAFLICWVPYASVAFYIFTHQGSNFGPIFMTLPAFFAKSAAIYNPVIYIMMNKQFRNCMITTLCCGKNPLGDDEASTTASKTETSQVAPA

SEQ ID NO: 6 - protein, 348 aa, Gorilla sp. [sequence omitted]

SEQ ID NO: 7 - protein, 1480 aa, Homo sapiens
  source 1..1480: note = cystic fibrosis transmembrane conductance regulator (CFTR)
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 8 - protein, 463 aa, Homo sapiens
  source 1..463: note = "Glucagon-like peptide 1 receptor (GLP-1R)"
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCS

SEQ ID NO: 9 - protein, 540 aa, Homo sapiens
  source 1..540: note = GPR75
MNSTGHLQDAPNATSLHVPHSQEGNSTSLQEGLQDLIHTATLVTCTFLLAVIFCLGSYGNFIVFLSFFDPAFRKFRTNFDFMILNLSFCDLFICGVTAPMFTFVLFFSSASSIPDAFCFTFHLTSSGFIIMSLKTVAVIALHRLRMVLGKQPNRTASFPCTVLLTLLLWATSFTLATLATLKTSKSHLCLPMSSLIAGKGKAILSLYVVDFTFCVAVVSVSYIMIAQTLRKNAQVRKCPPVITVDASRPQPFMGVPVQGGGDPIQCAMPALYRNQNYNKLQHVQTRGYTKSPNQLVTPAASRLQLVSAINLSTAKDSKAVVTCVIIVLSVLVCCLPLGISLVQVVLSSNGSFILYQFELFGFTLIFFKSGLNPFIYSRNSAGLRRKVLWCLQYIGLGFFCCKQKTRLRAMGKGNLEVNRNKSSHHETNSAYMLSPKPQKKFVDQACGPSHSKESMVSPKISAGHQHCGQSSSTPINTRIEPYYSIYNSSPSQEESSPCNLQPVNSFGFANSYIAMHYHTTNDLVQEYDSTSAKQIPVPSV

SEQ ID NO: 10 - protein, 466 aa, Homo sapiens
  source 1..466: note = GIPR
MTTSPILQLLLRLSLCGLLLQRAETGSKGQTAGELYQRWERYRRECQETLAAAEPPSGLACNGSFDMYVCWDYAAPNATARASCPWYLPWHHHVAAGFVLRQCGSDGQWGLWRDHTQCENPEKNEAFLDQRLILERLQVMYTVGYSLSLATLLLALLILSLFRRLHCTRNYIHINLFTSFMLRAAAILSRDRLLPRPGPYLGDQALALWNQALAACRTAQIVTQYCVGANYTWLLVEGVYLHSLLVLVGGSEEGHFRYYLLLGWGAPALFVIPWVIVRYLYENTQCWERNEVKAIWWIIRTPILMTILINFLIFIRILGILLSKLRTRQMRCRDYRLRLARSTLTLVPLLGVHEVVFAPVTEEQARGALRFAKLGFEIFLSSFQGFLVSVLYCFINKEVQSEIRRGWHHCRLRRSLGEEQRQLPERAFRALPSGSGPGEVPTSRGLSSGTLPGPGNEASRELESYC

SEQ ID NO: 11 - protein, 317 aa, Homo sapiens
  source 1..317: note = MC1R
MAVQGSQRRLLGSLNSTPTAIPQLGLAANQTGARCLEVSISDGLFLSLGLVSLVENALVVATIAKNRNLHSPMYCFICCLALSDLLVSGSNVLETAVILLLEAGALVARAAVLQQLDNVIDVITCSSMLSSLCFLGAIAVDRYISIFYALRYHSIVTLPRARRAVAAIWVASVVFSTLFIAYYDHVAVLLCLVVFFLAMLVLMAVLYVHMLARACQHAQGIARLHKRQRPVHQGFGLKGAVTLTILLGIFFLCWGPFFLHLTLIVLCPEHPTCGCIFKNFNLFLALIICNAIIDPLIYAFHSQELRRTLKEVLTCSW

SEQ ID NO: 12 - protein, 297 aa, Homo sapiens
  source 1..297: note = MC2R
MKHIINSYENINNTARNNSDCPRVVLPEEIFFTISIVGVLENLIVLLAVFKNKNLQAPMYFFICSLAISDMLGSLYKILENILIILRNMGYLKPRGSFETTADDIIDSLFVLSLLGSIFSLSVIAADRYITIFHALRYHSIVTMRRTVVVLTVIWTFCTGTGITMVIFSHHVPTVITFTSLFPLMLVFILCLYVHMFLLARSHTRKISTLPRANMKGAITLTILLGVFIFCWAPFVLHVLLMTFCPSNPYCACYMSLFQVNGMLIMCNAVIDPFIYAFRSPELRDAFKKMIFCSRYW

SEQ ID NO: 13 - protein, 323 aa, Homo sapiens
  source 1..323: note = MC3R
MNASCCLPSVQPTLPNGSEHLQAPFFSNQSSSAFCEQVFIKPEVFLSLGIVSLLENILVILAVVRNGNLHSPMYFFLCSLAVADMLVSVSNALETIMIAIVHSDYLTFEDQFIQHMDNIFDSMICISLVASICNLLAIAVDRYVTIFYALRYHSIMTVRKALTLIVAIWVCCGVCGVVFIVYSESKMVIVCLITMFFAMMLLMGTLYVHMFLFARLHVKRIAALPPADGVAPQQHSCMKGAVTITILLGVFIFCWAPFFLHLVLIITCPTNPYCICYTAHFNTYLVLIMCNSVIDPLIYAFRSLELRNTFREILCGCNGMNLG

SEQ ID NO: 14 - protein, 332 aa, Homo sapiens [sequence omitted]

SEQ ID NO: 15 - protein, 325 aa, Homo sapiens
  source 1..325: note = MC5R
MNSSFHLHFLDLNLNATEGNLSGPNVKNKSSPCEDMGIAVEVFLTLGVISLLENILVIGAIVKNKNLHSPMYFFVCSLAVADMLVSMSSAWETITIYLLNNKHLVIADAFVRHIDNVFDSMICISVVASMCSLLAIAVDRYVTIFYALRYHHIMTARRSGAIIAGIWAFCTGCGIVFILYSESTYVILCLISMFFAMLFLLVSLYIHMFLLARTHVKRIAALPGASSARQRTSMQGAVTVTMLLGVFTVCWAPFFLHLTLMLSCPQNLYCSRFMSHFNMYLILIMCNSVMDPLIYAFRSQEMRKTFKEIICCRGFRIACSFPRRD

SEQ ID NO: 16 - protein, 477 aa, Homo sapiens
  source 1..477: note = GCGR
MPPCQPQRPLLLLLLLLACQPQVPSAQVMDFLFEKWKLYGDQCHHNLSLLPPPTELVCNRTFDKYSCWPDTPANTTANISCPWYLPWHHKVQHRFVFKRCGPDGQWVRGPRGQPWRDASQCQMDGEEIEVQKEVAKMYSSFQVMYTVGYSLSLGALLLALAILGGLSKLHCTRNAIHANLFASFVLKASSVLVIDGLLRTRYSQKIGDDLSVSTWLSDGAVAGCRVAAVFMQYGIVANYCWLLVEGLYLHNLLGLATLPERSFFSLYLGIGWGAPMLFVVPWAVVKCLFENVQCWTSNDNMGFWWILRFPVFLAILINFFIFVRIVQLLVAKLRARQMHHTDYKFRLAKSTLTLIPLLGVHEVVFAFVTDEHAQGTLRSAKLFFDLFLSSFQGLLVAVLYCFLNKEVQSELRRRWHRWRLGKVLWEERNTSNHRASSSPGHGPPSKELQFGRGGGSQDSSAETPLAGGLPRLAESPF

SEQ ID NO: 17 - protein, 443 aa, Homo sapiens
  source 1..443: note = DRD2
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 18 - protein, 471 aa, Homo sapiens
  source 1..471: note = HTR2A
MDILCEENTSLSSTTNSLMQLNDDTRLYSNDFNSGEANTSDAFNWTVDSENRTNLSCEGCLSPSCLSLLHLQEKNWSALLTAVVIILTIAGNILVIMAVSLEKKLQNATNYFLMSLAIADMLLGFLVMPVSMLTILYGYRWPLPSKLCAVWIYLDVLFSTASIMHLCAISLDRYVAIQNPIHHSRFNSRTKAFLKIIAVWTISVGISMPIPVFGLQDDSKVFKEGSCLLADDNFVLIGSFVSFFIPLTIMVITYFLTIKSLQKEATLCVSDLGTRAKLASFSFLPQSSLSSEKLFQRSIHREPGSYTGRRTMQSISNEQKACKVLGIVFFLFVVMWCPFFITNIMAVICKESCNEDVIGALLNVFVWIGYLSSAVNPLVYTLFNKTYRSAFSRYIQCQYKENKKPLQLILVNTIPALAYKSSQLQMGQKKNSKQDAKTTDNDCSMVALGKQHSEEASKDNSDGVNEKVSCV

SEQ ID NO: 19 - protein, 2273 aa, Homo sapiens [sequence omitted]

SEQ ID NO: 20 - protein, 13 aa, synthetic construct
  source 1..13: note = Full length, TCS(S)
GSENLYFQSGSSY

SEQ ID NO: 21 - protein, 11 aa, synthetic construct
  source 1..11: note = Full length, TCS(Y)
GSENLYFQYGS

SEQ ID NO: 22 - protein, 7 aa, synthetic construct
  source 1..7: note = Protease cleavage site
ENLYFQX

SEQ ID NO: 23 - protein, 7 aa, synthetic construct
  source 1..7: note = Protease cleavage site, TCS(S)
ENLYFQS

SEQ ID NO: 24 - protein, 7 aa, synthetic construct
  source 1..7: note = Protease cleavage site, TCS(Y)
ENLYFQY

SEQ ID NO: 25 - protein, 90 aa, synthetic construct
  source 1..90: note = zinc finger (RARR)
ARPGERPFQCRICMANFSRNFILQRHTRTHTGEKPFQCRICMRNFSDRANLRRHLRTHTGEKPFQCRICMRNFSRHDQLTRHLKTHLRGS

SEQ ID NO: 26 - protein, 147 aa, Saccharomyces cerevisiae
  source 1..147: note = gal4
MKLLSSIEQACDICRLKKLKCSPEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 27 - protein, 162 aa, Saccharomyces cerevisiae
  source 1..162: note = PPR1
MKSRTACKRCRLKKIKCDQEFPSCKRCAKLEVPCVSLDPATGKDVPRSYVFFLEDRLAVMMRVLKEYGVDPTKIRGNIPATSDDEPFDLKKYSSVSSLGEEGILPHNGLLADYLVQKGNSMASSAITSKSMASPQTINVQRKEFLVNSKKQDGSALLPETGS

SEQ ID NO: 28 - protein, 146 aa, Saccharomyces cerevisiae
  source 1..146: note = Lac9
MNKKSSEVMHQACKRCRLKKIKCDQEFPSCKRCLKYNLDCVYSPQVVRTPLTRAHLTEMENRVAELEQFLKELFPVWDIDRLLQQKDTYRIRELLTMGSTNTVPGLASNNIDSSLEQPVAFGTAQPAQSLSTDPAVQSQAYPMQPV

SEQ ID NO: 29 - protein, 202 aa, Escherichia coli
  source 1..202: note = LexA
MKALTARQQEVFDLIRDHISQTGMPPTRAEIAQRLGFRSPNAAEEHLKALARKGVIEIVSGASRGIRLLQEEEEGLPLVGRVAAGEPLLAQQHIEGHYQVDPSLFKPNADFLLRVSGMSMKDIGIMDGDLLAVHKTQDVRNGQVVVARIDDEVTVKRLKKQGNKVELLPENSEFKPIVVDLRQQSFTIEGLAVGVIRNGDWL

SEQ ID NO: 30 - protein, 50 aa, synthetic construct
  source 1..50: note = VP64
DALDDFDLDMLGSDALDDFDLDMLGSDALDDFDLDMLGSDALDDFDLDML

SEQ ID NO: 31 - protein, 554 aa, synthetic construct
  source 1..554: note = VPr
GKPIPNPLLGLDSTDALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFNSACKNWFSSLSHFVIHL

SEQ ID NO: 32 - protein, 265 aa, Homo sapiens
  source 1..265: note = p65
QYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 33 - protein, 190 aa, unidentified
  source 1..190: note = Rta
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 34 - protein, 11 aa, unidentified
  source 1..11: note = VP16
DALDDFDLDML

SEQ ID NO: 35 - protein, 14 aa, Homo sapiens
  source 1..14: note = Lyn11
MGCIKSKGKDSAGA

SEQ ID NO: 36 - protein, 125 aa, Homo sapiens
  source 1..125: note = PHDelta1 Pleckstrin Homology domain-1
MHGLQDDEDLQALLKGSQLLKVKSSSWRRERFYKLQEDCKTIWQESRKVMRTPESQLFSIEDIQEVRMGHRTEGLEKFARDVPEDRCFSIVFKDQRNTLDLIAPSPADAQHWVLGLHKIIHHSGS

SEQ ID NO: 37 - protein, 111 aa, Homo sapiens
  source 1..111: note = PHDelta3 Pleckstrin Homology domain-3
MRAMLRGSRLRKIRSRTWHKERLYRLQEDGLSVWFQRRIPRAPSQHIFFVQHIEAVREGHQSEGLRRFGGAFAPARCLTIAFKGRRKNLDLAAPTAEEAQRWVRGLTKLRA

SEQ ID NO: 38 - protein, 49 aa, Homo sapiens
  source 1..49: note = platelet-derived growth factor receptor
AVGQDTQEVIVVPHSLPFKVVVISAILALVVLTIISLIILIMLWQKKPR

SEQ ID NO: 39 - protein, 12 aa, Synthetic construct
  source 1..12: note = ASPS
ASPSNPGASNGS

SEQ ID NO: 40 - protein, 10 aa, synthetic construct
  source 1..10: note = 2xGS
GGGGSGGGGS

SEQ ID NO: 41 - protein, 240 aa, unidentified
  source 1..240: note = Tobacco Etch Virus (TEV)
SLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNELVYSQ

SEQ ID NO: 42 - protein, 240 aa, unidentified
  source 1..240: note = Tobacco Etch Virus, S219V (TEV-S219V)
SLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNELVYSQ

SEQ ID NO: 43 - protein, 83 aa, synthetic construct
  source 1..83: note = YB_tata
GCGATTAATCCATATGCTCTAGAGGGTATATAATGGGGGCCACTAGTCTACTACCAGAAAGCTTGGTACCGAGCTCGGATCCA

SEQ ID NO: 44 - protein, 386 aa, Synthetic construct
  source 1..386: note = Dox-inducible
TCCCTATCAATGATAGAGAGGTTATTAAGACACCTCTTCCCTATCACTGATAGAGAACTCGACACGACTGGAGCTCCCTATCAGTGATAGAGAAGGTACGTCAAGAACGACTCCCTATCATTGATAGAGAAGGACTGTCTAAGACGACTCCCTATCAATGATAGAGAACTCGACAGTACGCTAAGTCCCTATCACTGATAGAGACCTGAAGGAGTCTACACATCCCTATCAGTGATAGAGAGCGCTTAATCGACAGAAGTCCCTATCATTGATAGAGAAGGAACCCCCTATATAAGCAGAGCTCGTTTAGTGAACCGTCAGATCGCCTGGAGTCGCCATCCACGCTGTTTTGACCTCCATAGATGACACCGGGACCGATCCAGCCT